(19) United States
(12) Patent Application Publication
(10) Pub. No.: US 2005/0053625 A1
(43) Pub. Date: Mar. 10, 2005

(54) Method of treating viral infections

(76) Inventors: Timothy M Block, Doylestown, PA (US); Anand Mehta, Lansdale, PA (US); Raymond Dwek, Oxford (GB)

Correspondence Address: David S Resnick;Nixon Peabody, 100 Summer Street, Boston, MA, 02110-2131, US

(21) Appl. No.: 10/494,377

(22) PCT Filed: Oct. 30, 2002
(86) PCT No.: PCT/US02/34732

Related U.S. Application Data
(60) Provisional application No. 60/339,191, filed on Oct. 30, 2001.

(51) Int. Cl. A61K 39/29

(52) U.S. Cl. 424/225.1; 424/227.1; 424/228.1

(57) ABSTRACT

The present invention relates to the treatment of viral infections, particularly HBV and HCV infections, with a combination comprising a vaccine against a virus antigen and compounds that inhibit glucosidase activity in the host cell.

### BACKGROUND OF THE INVENTION

The present invention relates to the treatment of viral infections, particularly HBV and HCV infections, with a combination comprising a vaccine against a virus antigen and compounds that inhibit glucosidase activity in the host cell.

Hepatitis B virus (HBV) disease remains a major world problem. There is a clear need for the introduction of safe and effective therapies for chronic hepatitis B virus (HBV). Worldwide, more than 350 million people are chronically infected with HBV and between 15 and 40% of these individuals will die from serious liver diseases, if left untreated. (Robinson, 1990). The major complication is the development of primary hepatocellular carcinoma (HCC) which causes an estimated 500,000 deaths annually (Beasley, 1988).

Currently there is no definitive cure for chronic HBV infection. However, several clinical approaches that have been beneficial in some individuals and there are currently two US FDA approved drugs for treatment of chronic HBV. Alpha-interferon, an immuno modulator, has been shown to be effective in achieving certain serological milestones in between 7-40% of treated patients (Hollinger, 1990). However, the need for parenteral administration, the poor long term response along with the high frequency of adverse side effects makes interferon non-ideal (Hoofnagle & Di Bisceglie, 1997). The other FDA approved HBV treatment is a nucleoside analogue, “3TC”, now sold as lamivudine/Epivir HBV.” It is effective against human immunodeficiency virus (HIV), as well as HBV, and its mechanism of action is well understood: it is a competitive inhibitor of the viral reverse transcriptase (Hoofnagle & DiBisceglie, 1997). Unlike interferon, it is orally available and effective in reducing viremia in almost all patients (Tipples et al., 1996; Mason et al., 1998). However, constitutive therapy is necessary and, unfortunately, escape mutants that have gained resistance to lamivudine occur 10-20% of the treated, per year. However, more treatment methods to treat HBV infections would be desirable.

In addition to HBV infections, more than 40 million people worldwide are chronically infected with the hepatitis C virus (HCV), and this represents one of the most serious threats to the public health of developed nations (Hoofnagle et al. (1997) New Engl J Med 336:347-356). Hepatitis C infection is the cause of more than 10,000 deaths annually in the United States (Hepatitis C Treatment, Washington Post, Nov. 11, 1997, at A2), a number that is expected to triple in the next twenty years in the absence of effective intervention. Chronic HCV also increases the risk of liver cancer. There are more than 40 million people worldwide who are chronically infected with HCV, representing one of the most serious threats to the public health of developed nations (Hoofnagle et al. (1997) New Engl J Med 336:347-356). Persistent infection develops in as many as 85% of HCV patients and in at least 20% of these patients the chronic infection leads to cirrhosis within twenty years of onset of infection. With an estimated 3.9 million North Americans chronically infected, complications from Hepatitis C infection is now the leading reason for liver transplantation in the United States.

HCV is an RNA virus belonging to the Flaviviridae family. Individual isolates consist of closely related, yet heterologous populations of viral genomes. This genetic diversity enables the virus to escape the host's immune system, leading to a high rate of chronic infection.

Therapeutic interventions which are effective for treatment of HCV infection are limited in number and effectiveness. Standard treatment for HCV infection includes administration of interferon-alpha. However, interferon-alpha is of limited use in about 20% of the HCV-infected population (Hoofnagle et al. (1997) New Engl J Med 336:347-356) and treatment with this compound results in long-term improvement in only 5% of patients. Furthermore, the complications and limitations of interferon-alpha seriously limit the applicability of the treatment. An experimental treatment comprising administration of interferon-alpha and ribavirin (1-.alpha.-D-ribofuranosyl-1H-1,2,4-triazole-3-carboxamide) resulted in long-term improvement in only half of patients suffering a relapse of HCV infection (Hepatitis C Treatment Washington Post, Nov. 11, 1997, at A2). Clearly, the disappointing results with interferon must prompt a search for more effective and less toxic therapeutics. Thus, a critical need remains for a therapeutic intervention that effectively treats HCV infection.

A large number of individuals who are infected with HCV are also infected with HBV. The therapy for combined HBV/HCV infection is particularly challenging because the HBV and HCV viruses differ from one another in therapeutically significant ways. HBV is a hepadnavirus, while HCV is a pestivirus. HBV is a DNA-containing virus, the genome of which is replicated in the nucleus of the infected cell using a combination of a DNA-dependent RNA polymerase and an RNA-dependent DNA polymerase (i.e., a reverse transcriptase). HCV is an RNA-containing virus, the genome of which is replicated in the cytoplasm of the infected cell using one or more types of RNA-dependent RNA polymerases. Despite the frequent concurrence of HBV infection and HCV infection, a number of compounds known to be effective for treating HBV infection are not effective against HCV. For example, lamivudine (the nucleoside analog 3TC) is useful for treating HBV infection, but is not useful for treating HCV infection. The difference in the susceptibility of HBV and HCV to antiviral agents no doubt relates to their genetically based replicative differences. There remains a particularly critical need for a therapeutic intervention that effectively treats both HBV and HCV infection.

Animal viruses that acquire their envelope from a membrane associated with the intracellular membrane of an infected animal cell cause significant losses to the livestock industry (Sullivan et al. (1995) Virus Res 38:231-239). Such animal viruses include pestiviruses and flaviviruses such as bovine viral diarrhea virus (BVDV), classical swine fever virus, border disease virus and hog cholera virus.

The flavivirus group to which HCV belongs is known to include the causative agents of numerous human diseases transmitted by arthropod vectors. Human diseases caused by flaviviruses include various hemorrhagic fevers, hepatitis, and encephalitis. Viruses known to cause these diseases in humans have been identified and include, for example, yellow fever virus, dengue viruses 1-4, Japanese encephalitis virus, Murray Valley encephalitis virus, Rocio virus, West Nile fever virus, St. Louis encephalitis virus, tick-borne encephalitis virus, Louping ill virus, Powassan virus, Omsk hemorrhagic fever virus, and Kyasanur forest disease virus.

Therefore, it would be desirable to develop treatment methods that would provide an effective treatment for both HBV and HCV infections.

### SUMMARY OF THE INVENTION

The present invention capitalizes on a unique strength of glucosidase inhibitors: their ability to reduce antigen/glycoprotein secretion. Current therapeutic approaches for the treatment of HBV and/or HCV only rarely reduce antigenemia (S, M, or LHBs in the circulation). Reductions in antigenemia are thought to be largely a secondary consequence of reductions of viremia, limiting reinfection mediated spread of the virus, and require very long period of treatment (Nowak et al., 1998). We have shown, in tissue culture and in the woodchuck model, that safe and well tolerated doses of glucosidase inhibitor can reduce the amount of M HBV antigen secretion, selectively, within days (in tissue culture) and weeks (in woodchucks). Without wishing to be bound by theory, we believe that therapeutic vaccination of HBV carriers with HBV glycoproteins can be enhanced when antigenemia is concomitantly reduced, and much of the stimulation of the cellular immune response was directed against the MHBs (preS2) antigen epitopes.

The present invention provides a method of treating an HBV and/or HCV infection in a subject by combining vaccination of the subject with a virus antigen comprising vaccine and administering to the subject an agent which inhibits morphogenesis of a virus which acquires its envelope from a membrane-associated with the intracellular membrane of an infected cell.

In one embodiment, the invention provides a method of treating a subject infected with a virus that is characterized by acquiring its envelope from a membrane associated with the ER of a virus-infected cell. The method comprises administering to a subject in need thereof a viral antigen comprising vaccine and administering to the subject a glucosidase inhibitor in an amount effective to inhibit the activity of a glucosidase enzyme with the endoplasmic reticulum of a virus-infected cell of the animal, thereby reducing, ablating, or diminishing the virus infection in the animal. The animal is preferably a mammal such as a pig or a cow and, particularly, a human being.

In one embodiment, the invention includes a method vaccinating a subject with a vaccine comprising an antigen from an HBV and/or HCV and inhibiting morphogenesis of a virus that acquires its envelope from an internal cell membrane associated with the endoplasmic reticulum (ER). The method comprises administering to a subject in need thereof an HBV and/or HCV vaccine and administering to the subject a glucosidase inhibitor in an amount effective to inhibit the activity of a glucosidase enzyme associated with the endoplasmic reticulum of the cell, thereby inhibiting morphogenesis of the virus. Mammalian cells infected with the subject viruses including, but not limited to, human liver cells and bovine monocytes are particularly contemplated as therapeutic targets.

The methods of the invention are useful for treating subjects with infections associated with viruses, such as HBV and HCV, that acquire their envelope from a membrane associated with the ER inhibiting morphogenesis of a virus. Because both flaviviruses and pestiviruses acquire their envelopes from membranes associated with the ER, the methods of the invention are contemplated to be particularly useful for inhibiting morphogenesis of, or for treatment of infection by flaviviruses and pestiviruses.

In another embodiment, the invention provides a method for vaccinating a subject with a virus antigen comprising vaccine and targeting a glucosidase inhibitor or glucosyltransferase inhibitor to the liver cell of an animal by targeting said liver cells with an N-alkyl derivative of a 1,5-dideoxy-1,5imino-D-glucitol. In a preferred embodiment the derivative is an N-nonyl-1,5-dideoxy-1,5-imino-D-glucitol.

In yet another embodiment, the invention provides a prophylactic method for protecting a subject infected by a virus that acquires a viral component from an internal membrane of an animal cells from developing a cancer that is among the sequelae of infection by said virus, comprising administering to the virus infected cell of the animal an effective anti-viral amount of an animal cell glucosidase-inhibitor. In a preferred embodiment of this aspect of the invention the antiviral glucosidase inhibitor is selected from the group consisting of 1,5-dideoxy-1,5-imino-D-glucitol and derivatives thereof.

### BRIEF DESCRIPTION OF THE FIGURES

FIG. 1 is a schematic presentation of glycoprotein genes and gene products of hepatitis B virus (HBV). The open box shows the overlapping reading frames for LHBs, MHBs and SHHBs, with their respective AUG translational start sites, which are highlighted. Solid boxes represent the contiguous polypeptide gene product(s) with the bars to the right inducating how the polypaptides would appear to migrate in SDS polyacrylamide gels and the apparent molecular weights of the glycosylated (gp) or unglycosylated (p) are also provided.

FIG. 2 shows N-linked glycan processing in the endoplasmic reticulum (ER). The 13 sugar residue oligosaccharide structure (shown as a pitch fork with three circles) is added to nascent polypeptides (shown as a ribbon) in the ER by the action of oligosaccharyl transferase (OST) at specific asparagine residues. This step is blocked by tunicamycin (Tun). Immediately after transfer to the polypeptide, terminal glucose residues (shown as open circles) are sequentially removed from the N-linked glycan oligosaccharide by the ER glucosidases (Glu), which are inhibited by prototype imino sugar deoxnojirymicins we call “glucovirs” (DNJ, NB-DNJ and NN-DNJ in the Examples). The ER chaperon, calnexin is a lectin that binds the nascent polypeptide via its monoglucose residue and mediates proper folding (note the polypeptide structure is altered in the figure following calnexin binding). Glucosidase II removes the final glucose, and mannosidases, which are inhibited by deoxymanojirimycin (DMJ) and is then transferred to the Golgi apparatus for further processing and complex carbohydrate formation. (See, Rudd and Dwek (1997) and Elbein (1991) for details).

FIG. 3 shows secretion of virus from Hep G2.2.15 cells after treatment with glucosidase inhibitor. Virus secretion into the media was detected by a method that would differentiate between enveloped and un-enveloped DNA. Glucosidase inhibitors reduce the secretion of enveloped virus. Enveloped virus is resistant to Proteinase K/Dnase treatment; un-enveloped virus is not. (+) or (−) indicates the addition of DNase. See text for more details. From left to right: Untreated; 3TC (3.5 μM); DNJ (4.5 mM).

FIG. 4 shows secretion of the M only sub-viral particles in the presence of the indicated imino sugars. Hep G2 cells were transfected with an M only expression vector and the next day the cells divided equally into 6 well trays. Compound was added one day latter at the indicated concentrations and the media changed every two days. The presence of sub-viral particles in the medium was detected via the Abbott Diagnostics Auszyme Monoclonal Diagnostic Kit as per manufacture's directions. For Graph: Y-axis is the OD at 492. X-axis indicates the compounds or cells used in the assay.

FIG. 5 demonstrates that glucosidase inhibition causes the accumulation of the HBV M protein. Hep G2 cells transfected with the HBV M expression vector were treated with the glucosidase inhibitor NB-DNJ (4.5 mM and 6 days latter the amount of M protein associated with the culture medium (secreted) and the cells (retained) determined by ELISA. See text for more details. For graph: x-axis, Drug treatment and location of protein; for y-axis, % of HBV M protein as compared to untreated

FIG. 6 shows that M protein secretion remains depressed long after glucosidase inhibitor removal. Hep G2 cells were transfected with the M only expression vector and seeded as daughter flasks into 6 well trays. Following 4 days of treatment of glucosidase inhibitor (NB-DNJ (4.5 mM)), drug was either removed (in samples labels as —R) or left on as a control. Day 0 is the time in which drug was removed. Hence NB—R is in the presence of compound. Day 3 and Day 5 are days after drug removal. For Graph: Y-axis is the OD at 492. X-axis indicates the compounds or cells used in the assay. UN=untreated sample; Un-R is the untreated rebound sample; NB=N-butyl-DNJ; NB-R=N-butyl-DNJ rebound sample.

FIG. 7 demonstrates that N-nonyl-DNJ treated animals do not secrete the M protein. 300 μl of serum from untreated (M301) or N-nonyl-DNJ treated (F363) animals either before treatment (0) or 3 weeks after treatment (3) were partially purified through 20% sucrose s and the proteins resolved through SDS polyacrylamide gels (12.5%). WHV M protein was detected by immunoblot. The WHV M protein band is indicated. All samples were analyzed by Anthony Willis (MRC Unit, University of Oxford) by N-terminal sequence analysis. The WHV M protein differs from the human HBV M protein due to extensive O-linked glycan modification (Toll et al., 1999).

FIG. 8 shows an example of some of glucosidase inhibitors available to use. All of the glucosidase inhibitors used in this study are based upon the DNJ heard group. Modification of the tail can both increase efficacy and potency while decreasing toxicity. The relative CC50 and IC50 values for BVDV and HBV are given as an example.

FIGS. 9A-9D show kinetics of serum anti-WHs antibody response (U/ml) from chronic WHV carrier woodchucks from 4 experimental groups. L+V+, L—FMAU +Vaccine treated animals; L+V−, F—FMAU treated animals only, L−V+, Vaccine treated animals only, L−V−, untreated animals. The drug treatment period is indicated. Vaccination occurred at 32 weeks and every 4-8 weeks thereafter (Taken from Menne et al., 2002).

### DETAILED DESCRIPTION OF THE INVENTION

The present invention provides a method of treating an HBV and/or HCV infection in a subject by combining vaccination of the subject with a virus antigen comprising vaccine and administering to the subject an agent which inhibits morphogenesis of a virus which acquires its envelope from a membrane-associated with the intracellular membrane of an infected cell. Without wishing to be bound by theory, it is believed that vaccination against the virus antigens combined with inhibition of virus morphogenesis effectively reduces the HBV and HCV infection.

Viral hepatitis is a chronic necroinflammatory disease, with the infected host playing a critical role in pathogenesis (Chisari, 2000). The cellular immune response to HBV, in the infected individual, appears to be involved in both the clearance of virus as well as in mediating chronic disease (Chisari, 2000; Guidotti; Webster & Bertoletti, 2001). The disease course may be governed by the quality (specificity) and robustness of the cellular response (Rehermann, et al, 1996; Webster & Bertolitti, 2001). Although some level of immunological control is thought to involve direct cell killing of infected cells, there is compelling experimental and clinical evidence that non cytotoxic immunological mechanisms can reduce the amount of viral gene product burden, as well. Replicative forms of HBV DNA within infected hepatocytes, in vivo, can be reduced by signals communicated to the infected hepatocytes via cytokines (Guidotti et al, 1999, Rehermann et al, 1996). Work with transgenic mice, bearing an expressed HBV transgene as well as chimpanzees, is consistent with the notion that activation of appropriate cell of the immune system (non killer CD8+ T cells or NKT cells, for example) results in a cytokine mediated reduction of HBV DNA levels within infected hepatocytes (Guidotti et al, 1999, Wieland et al, 2000). Significantly, the hepatocytes appear to remain viable (Guidotti et al 1999). There is also evidence that these events occur during natural infection of people (Rerhmann et al, 1996). The inflammatory cytokine mediators involved include interferon alpha, TNF alpha and interleukin 2 (Wieland et al, 2000). Detailed analysis of the kinetics and biochemistry of the disappearance of HBV gene products in transgenic mice following induction of inflammatory cytokines suggests that viral transcription and polypeptide production and secretion rate is not, at least initially, significantly influenced, implicating either assembly or degradation of pregenomic RNA nucleocapsids or degradation (Wieland et al 2000).

Tissue cultures of hepatocyte-derived cells bearing HBV transgenes incubated with interferon alpha experience reductions in intracellular forms of HBV DNA, without significant impacts upon viral gene product production or secretion, as well (Hayashi & Koike, 1989). These data, along with that described above, demonstrate that hepatocytes have the cell machinery to greatly and specifically reduce intracellular HBV DNA levels without a detectable impact upon cellular DNA or other macromolecular synthesis. The mechanisms mediating these processes are unclear, and interest is all the greater since the HBV DNA that is affected is extranuclear (in the cytoplasm or the ER). An understanding of the mechanisms by which the cell can “manage” HBV DNA, in this noncytolytic way, will provide enormous insights about innate cellular defense systems.

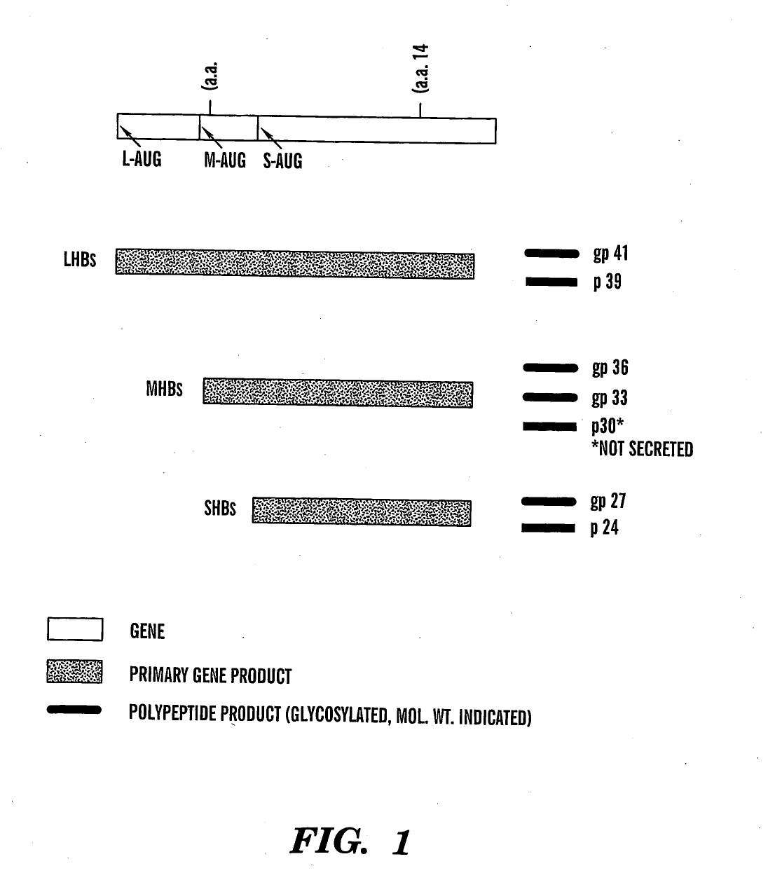

There is a growing body of evidence that animals and people chronically infected with hepadnaviruses possess the potential to mount a beneficial immunological responses to the virus. It appears that this response, in experimental animals and in chronically infected people, can be enhanced or enabled by either highly effective and sustained antiviral therapy or vaccination using hepadna viral antigens (Boni, C., et al 1998; Couillin, et al, 1999). As shown in FIG. 1. the HBV envelope is composed of three glycoproteins, called LHBs (L), MHBs (M) and SHBs (S) which are derived from alternative translation starts from the same open reading frame (Heerman and Gerlich, 1992) and are characterized by preS 1, preS2 and S domains, respectively. Of particular note was that in both the woodchuck and human study in which a preS2 containing vaccine was used, the majority of cellular responsiveness was to preS2 epitopes, and this will be addressed in greater detail, below.

Woodchuck hepatitis virus (WHV) is a naturally occurring hepadnavirus pathogen of woodchucks that shares many biochemical properties with human HBV (Tennant et al., 1994). Indeed, woodchucks with chronic WHV infection are recognized to be good animal models to test anti-viral agents with potential for treating the human disease. Nucleoside analogues, displaying efficacy against WHV in woodchucks have been shown to be effective against human HBV in clinical settings (Korba et al., 1996; Menne & Tennant, 1999). The ability of vaccination with sub viral sAg particles, derived from the serum of chronically infected animals, in combination with antiviral therapy, in eliciting a beneficial immunological response in woodchucks chronically infected with WHV has been demonstrated (Menne et al, 2002, appendix, and prelim. evidence). In this study, a potent anti-viral compound, L-FMAU (1-(2-Fluro-5-Methy-B-L-Arabinofuranosyl)-Uracil) was used to inhibit WHV replication. Long term treatment with this compound also resulted in a 10 fold decrease in the amount of serum WHsAg. Surprisingly, this reduction was associated with the return of both a humoral and a cellular immune responses. It is unclear if or why reduction in antigen burden facilitates humoral and cellular antigen recognition, but reductions of viremia by at least two orders of magnitude, alone, in the absence of reductions in antigenemia, in woodchucks, is not associated with a return of humoral or cellular immunity (unpublished observations). It is this area of interest and possibility that our proposal will address. The addition of a therapeutic vaccination dramatically increased the number of animals that developed beneficial immune responses to WHV as well as increasing the degree of response. Indeed, many animals, after anti-viral and therapeutic vaccination, responded similarly to animals with self-limiting WHV infections. This would imply the high viral antigen load is important in the maintenance of the chronic infection and thus the reduction of circulating antigen (sAg) would allow for the return of an immunological response and provide clinical benefit. In this case, vaccination may act to “boost” the response and allow for a more robust cellular response. It is noted that only animals that received both vaccine and drug treatment (antigen reduction) had significant beneficial immunological responses, reasoning that vaccine therapy may need a complementation.

Infections by viruses that require host cell glycosidase enzymes to synthesize and properly fold viral envelope glycoproteins can be treated by administering an inhibitor of those enzymes to the host cell. A target virus is any virus that acquires a component of its envelope in cooperation with internal cell membrane associated with the endoplasmic reticulum (ER). Preferred viruses are members of the hepabdna virus, flavivirus or pestivirus class.

By a “membrane associated with the ER” of a cell is meant a membrane which surrounds the lumen of the ER of the cell, a membrane which surrounds a lumen of the Golgi apparatus (GA), a membrane which surrounds the lumen of a vesicle passing from the ER to the GA, a membrane which surrounds the lumen of a vesicle passing from the GA to the ER, a membrane which surrounds the lumen of a vesicle passing from the GA or the ER to the plasma membrane of the cell, a membrane which surrounds the lumen of a vesicle passing from the GA or the ER to the nuclear membrane of the cell, or a membrane which surrounds the lumen of a vesicle passing from the GA or the ER to a mitochondrial membrane of the cell. It is contemplated that the methods of the invention are preferably applied to inhibiting the production of a virus that acquires any morphogenetic component by derivation from any of the internal membranes of the host cell.

By a “glucosidase enzyme associated with the ER” of a cell is meant a glucosidase enzyme which is embedded within, bound to the luminal side of, or contained within a membrane associated with, the ER of the cell. By way of example, mammalian .alpha.-glucosidase I and mammalian .alpha.-glucosidase II are glucosidase enzymes associated with the ER of a mammalian cell.

A virus-infected animal cell which is treated according to the methods of the invention may be any cell that comprises a glucosidase enzyme associated with an internal membrane of the cell, preferably an enzyme associated with the endoplasmic reticulum (ER). Treatment of mammalian cells, including but not limited to human liver cells and bovine monocytes are particularly preferred.

Agents that exhibit an inhibitory effect on glucosidases are believed to do so because they are structural analogs of glucose. One of these agents is the imino sugar designated 1,5-dideoxy-1,5-imino-D-glucitol (alternately designated deoxynojirimycin), hereinafter “DNJ.” Numerous DNJ derivatives have been described. DNJ and its alkyl derivatives are potent inhibitors of the N-linked oligosaccharide processing enzymes, .alpha.-glucosidase I and .alpha.-glucosidase II (Saunier et al. (1982) J Biol Chem 257:14155-14161; Elbein (1987) Ann Rev Biochem 56:497-534). These glucosidases are associated with the endoplasmic reticulum of mammalian cells. The N-butyl and N-nonyl derivatives of DNJ may also inhibit glucosyltransferases associated with the Golgi.

Methods for treating a mammal infected with respiratory syncytial virus (RSV) using DNJ derivatives have been described (U.S. Pat. No. 5,622,972 issued to Bryant et al.). It is believed that DNJ exhibits its inhibitory effects on glucosidase because it is a glucose analog.

The use of DNJ and N-methyl-DNJ has also been disclosed to interrupt the replication of non-defective retroviruses such as human immunodeficiency virus (HIV), feline leukemia virus, equine infectious anemia virus, and lentiviruses of sheep and goats (U.S. Pat. Nos. 5,643,888 and 5,264,356; Acosta et al. (1994) Am J Hosp Pharm 51:2251-2267).

Human Hepatitis B virus (HBV) secretion from human hepatoblastoma cells in tissue culture is sensitive to inhibitors of the a-glucosidase activity in the endoplasmic reticulum (ER) under conditions that do not compromise host viability (Block et al. 1994). Hepatitis B virus (HBV) infected liver cells secrete infectious, nucleocapsid-containing virions as well as an excess of non-infectious “subviral” articles that do not contain DNA. All of these particles are believed to bud from an ER compartment or a post-ER compartment such as the intermediate compartment (Huovila et al. (1992) J Cell Biol 118:1305-1320; Patzer et al. (1986) J Virol 58:884-892). Inhibition of mature HBV egress is caused by inhibition of the activity of one or more of the glucosidase enzymes or glucosyltransferase enzymes normally associated with the endoplasmic reticulum (ER) of 2.15 cells, which are derived from HepG2 cells (Lu et al. (1995) Virology 213:660-665; Lu et al. (1997) Proc Natl Acad Sci USA 94:2380-2385).

Studies suggest that the anti-HIV properties of DNJ derivatives are the result of improper glycoprocessing of HIV envelope proteins, rather than direct inhibition of HIV budding from cells (Dedera et al. (1990) AIDS Res Hum Retr6vir 6:785-794; Fischer et al. (1995) J Virol 69:5791-5797; Taylor et al. (1994) Antimicrob Agents Chemother 38:1780-1787).

One derivative of DNJ, namely N-butyl-1,5-dideoxy-1,5-imino-D-glucitol (NBDNJ), prevents egress of the mature HBV virion from stable transfected HepG2 cells, but does not prevent egress of subviral particles (Block et al. (1994) Proc Natl Acad Sci USA 91:2235-2239). Thus, morphogenesis of HIV virions which are believed to bud through the plasma membrane, appears to be unaffected by the presence of NBDNJ.

However, the infectivity of the virus particles released from HIV-infected cells exposed to NBDNJ is greatly reduced relative to HIV particles released from cells which were not exposed to NBDNJ (Dedera et al., supra Fisher et al., supra; Taylor et al., supra). These studies suggest that the anti-HIV properties of NBDNJ are the result of improper viral fusion of target cells, rather than direct irihibition of HIV budding from cells.

More recently we demonstrated the anti-viral effect of glucosidase inhibitors in a woodchuck animal model of HBV infection. In woodchucks chronically infected with woodchuck hepatitis virus (WHV), treatment with ER .alpha.-glucosidase inhibitors results in the disruption of the proper folding and transport of viral envelope glycoproteins and prevents the secretion of infectious enveloped virus (Block et al., 1998).

Most significantly and apparently different from the situation with HIV and RSV, inhibition of only modest amounts of glucosidase resulted in massive inhibition of HBV and BVDV secretion. This suggests that, unlike with HIV and RSV, etc., for viruses that bud from internal membranes, disruption of only a minority of envelope viral proteins is sufficient to inhibit secretion of the virus. This may be due to the fact that our evidence suggests that disrupted HBV and BVDV viral proteins act as “dominant negative” poisons of virus secretion and may themselves be considered the antiviral drug, as much as the drug itself.

ER α-glucosidases are responsible for the stepwise removal of terminal glucose residues from N-glycan chains attached to nascent glycoproteins. This enables the glycoproteins to interact with the ER chaperones calnexin and calreticulin, which bind exclusively to mono-glucosylated glyc proteins. Interaction with calnexin is crucial for the correct folding of some but not all glycoproteins, and inhibitors of the glucosidases can be used to specifically target proteins that depend on it. N-linked glycans play many roles in the fate and functions of glycoproteins. One function is to assist in the folding of proteins by mediating interactions of the lectin-like chaperone proteins calnexin and calreticulin with nascent glycoproteins. It is these interactions that can be prevented by inhibiting the activity of the alpha.-glucosidases with agents such as N-butyl-DNJ and N-nonyl-DNJ, causing some proteins to be misfolded and retained within the endoplasmic reticulum (ER). We have shown that the N-nonyl-DNJ-induced misfolding of one of the hepatitis B virus (HBV) envelope glycoproteins prevents the formation and secretion of virus in vitro and that this inhibitor alters glycosylation and reduces the viral levels in an animal model of chronic HBV infection.

The exquisite sensitivity of HBV to alterations in the envelope proteins induced by α-glucosidase inhibition and the fact that it is not necessary to inhibit the enzyme to any great extent in order to achieve the observed anti-viral effect, led us to speculate that the sensitivity of the virus may be due to the fact that it has to oligomerize and assemble the envelope in the ER where folding takes place. Unlike the situation with HIV and RSV, a few misfolded envelope proteins may be sufficient to disrupt the proper envelopment process and amplify the adverse effect the inhibitor has on virus assembly as compared to the effect it has on host cell proteins, which do not seem to be impaired at anti-viral inhibitor concentrations. Our mechanism studies led us to propose that other viruses which acquire their envelopes from intracellular membranes such as the ER would be equally sensitive to ER .alpha.-glucosidase inhibition, provided one or more of their glycoproteins depended on calnexin-mediated folding.

Although HBV and HCV have completely different life cycles, they have at least three aspects in common: (1) They target the liver, (2) they bud from the ER and other internal membranes and (3) their envelope, glycoprotein(s) fold via a calnexin-dependent pathway. This prompted us to investigate whether the same inhibitors shown to have an anti-viral effect on HBV could inhibit HCV by the same proposed mechanism.

The two HCV envelope glycoproteins E1 and E2, which contain five or six and eleven N-linked glycosylation sites, respectively, both interact with calnexin during productive folding (Choukhi et al., 1998). Due to the lack of an efficient cell culture replication system the understanding of HCV particle assembly is very, limited. However, the absence of complex glycans, the localization of expressed HCV glycoproteins in the ER, and the absence of these proteins on the cell surface suggest that initial virion morphogenesis occurs by budding into intracellular vesicles from the ER. Additionally, mature E1-E2 heterodimers do not leave the ER, and ER retention signals have been identified in the C-terminal regions of both E1 and E2.

This led us to investigate the effect of glucosidase inhibitors on another ER-budding virus, bovine viral diarrhea virus (BVDV), the tissue culture surrogate of human hepatitis C virus (HCV). In the absence of a suitable cell culture system able to support replication of human HCV, bovine viral diarrhea virus (BVDV) serves as the FDA approved model organism for HCV (FIG. 1), as both share a significant degree of local protein region homology (Miller et al., 1990), common replication strategies, and probably the same sub-cellular location for viral envelopment. Compounds found to have an antiviral effect against BVDV are highly recommended as potential candidates for treatment of HCV.

BVDV, like HCV, is a small enveloped positive-stranded RNA virus and, like all viruses within the Flaviviridae, encodes all of its proteins in a single, long open reading frame (ORF), with the structural proteins in the N-terminal portion of the polyprotein and the non-structural or replicative proteins at the C-terminal end. The BVDV polyprotein has 6 potential N-glycosylation sites in the region encoding for the two heterodimer-forming envelope proteins gp25 (E1) and gp53 (E2), and 8 potential N-glycosylation sites in the region encoding for gp48 (EO), a hydrophilic secreted protein of unknown function. The structures of the oligosaccharides attached to any of these glycoproteins remain to be determined. BVDV proved to be even more sensitive to ER .alpha-glucosidase inhibitors. This and the facts that the inhibitors used are preferentially taken up by liver-type cells in vitro and exhibit a prolonged retention in the liver in vivo give rise to the exciting possibility that glucosidase inhibitors could be used as broad based antiviral hepatitis agents.

We have discovered that cytotoxicity resulting from exposure of mammalian cells in tissue culture to bovine viral diarrhea virus (BVDV) is prevented by addition of a glucosidase inhibitor to the tissue culture medium. The glucosidase inhibitors useful according to the present invention include, but are not limited to a derivative of 1,5-dideoxy-1,5-imino-D-glucitol (DNJ), in particular, N-butyl-DNJ (NBDNJ). Moreover, inhibition of BVDV-induced cytotoxicity was achieved under conditions in which little, if any, toxicity toward host cells was observed to be mediated by NBDNJ. Because BVDV is an accepted tissue culture model of hepatitis C virus (HCV) (Henzler, H.-J. and K. Kaiser (1998) Nature Biotech 16:1077-1078), the compositions and methods described herein for inhibiting morphogenesis of BVDV are also useful for inhibiting morphogenesis of HCV.

Vaccines against both HBV and HCV exist but none of them have been able to provide reliable prevention the diseases associated with the HBV and HCV infections. Here we have discovered that by provoking a host immune response against a viral antigen and consequent treatment using glucosidase inhibitors, significantly improves the treatment of infection—the activated host immune response is more likely to succeed in overcoming the virus load when the virus load is reduced by glucosidase inhibitors.

Therefore, a goal of antiviral therapy that would most effectively complement the vaccine approach would be one that reduced circulating HBsAg (L, M and or S) levels in the blood. It is reasoned that if sustained reductions in antigenemia can be achieved, therapeutic vaccination will have the greatest chance of success.

We have previously shown that glucosidase inhibitors are effective in reducing the amount of HBV envelope proteins secreted from chronically infected cells (U.S. Pat. No. 6,465,487). Secretion of MHBs and LHBs are most sensitive to glucosidase inhibition, with SHBs much less so. Indeed, preliminary evidence suggests that as little as 15%-20% glucosidase inhibition can inhibit the secretion of the M protein. The limited observations made with woodchucks is reinforced by much more comprehensive studies we have performed in tissue culture. Clearly, the appearance of MHBs in the culture medium of HBV producing cells can be greatly reduced (or eliminated) by amounts of glucosidase inhibitor that have little, if any, adverse impact upon uninfected host cells.

Moreover, we have shown that the amount of glucosidase inhibitors (such as nonyl DNJ) needed to achieve dramatic reductions in the MHBs antigenemia in animals are well within a safe dose range.

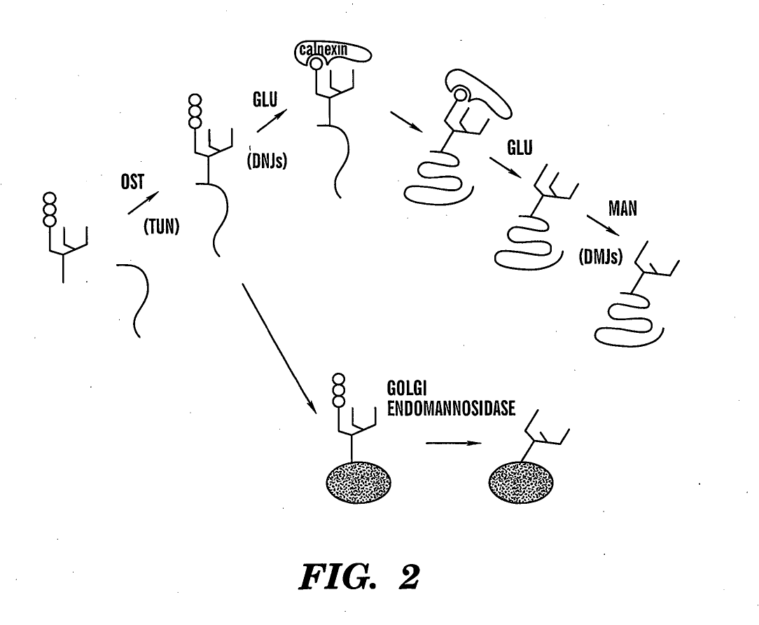

Glucosidase inhibitors prevent protein folding. Glucosidases I and II, in the endoplasmic reticulum (ER), mediate processing of N-linked glycan on glycoproteins. This processing is necessary for the interaction of many glycoproteins with the protein folding chaperon, calnexin, as shown in FIG. 2. The morphogenesis and secretion of members of the hepatitis B and flavivirus families are more dependent upon glucosidases (and presumably, calnexin) than are most cellular glycoproteins (Block & Jordan, 2002). We have speculated that viruses that acquire their envelope from ER or post ER compartments, such as hepadna and flaviruses, will be, as a class, dependent upon glucosidases to complete their life cycle. HBV M glycoprotein folding and secretion appears to have an obligate requirement for calnexin mediated folding and processing by ER glucosidases (Werr & Prange, 1997). HBV M (and to a large extent, L) glycoprotein secretion is prevented in glucosidase inhibited cells, with glycoprotein's accumulating within the intracellular compartment. Defective HBV M glycoproteins accumulate in glucosidase inhibited cells and may act in a dominant negative manner, antagonizing the secretion of future viral particles (Lu et al., 1997, Mehta et al., 1997).

Glucosidase inhibitors as antiviral agents. Since the inhibition of MHBs and HBV secretion can occur under conditions where glucosidase mediated glycoprocessing is only modestly affected, the use of glucosidase inhibitors as antiviral agents has been proposed (Mehta et al, 1997, Block & Jordan, 2002). It is emphasized that since glucosidase inhibitors are also effective against Dengue virus, in tissue culture (Courgetot et al, 2000) and Japanese Encephalitis Virus in tissue culture and in a the mouse model (Zitzmann, in press), the possibility their development as therapies for these diseases, which are of both great world health concern and bio-terrorism threats, has generated a new excitement.

Although secretion of HBV and members of the flavivirus family are certainly more sensitive to glucosidase inhibitors than are most cellular functions, high dose, long term inhibition of glucosidase can have adverse effects on the host, and the reduction of viremia is usually less than that achieved with standard antiviral agents that target components of the viral replication machinery. We, and others, have made considerable progress in developing glucosidase inhibitors that have reduced toxicity and improved efficacy, in culture and in vivo (animal studies). For example, alkylated imino sugars with side chains of greater than 8 carbons bearing either hexylations or methoxylations at their termini have greatly improved efficacy against HBV and BVDV, in tissue culture with reduced toxicity (Zitzmann et al, 2001; Mehta et al, 2002a,b). Acetylation or methylation of the head group sugar reduces gastrointestinal toxicity, in animals and people (Mehta et al, unpublished). There are thus logical chemistry strategies to improve the performance of these molecules (see Prelim. Evidence and appendix).

As an “antigen reduction” therapy, imino sugar glucosidase inhibitors may be unique. The focus of all previous antiviral efforts with these compounds has been upon their ability to reduce viremia in short term, mono therapeutic approaches. The present invention describes a unique strength of these inhibitors: their ability to reduce antigen/glycoprotein secretion. Current therapeutic approaches for the treatment of HBV and/or HCV only rarely reduce antigenemia (S, M, or LHBs in the circulation). Reductions in antigenemia are thought to be largely a secondary consequence of reductions of viremia, limiting reinfection mediated spread of the virus, and require very long period of treatment (Nowak et al., 1998). We have shown, in tissue culture and in (in a much more limited way, in woodchucks), that safe and well tolerated doses of glucosidase inhibitor can reduce the amount of M HBV antigen secretion, selectively, within days (in tissue culture) and weeks (in woodchucks). Under the same conditions, lamivudine had no detectable effect upon HBV antigen secretion, as expected, although viral replication was reduced to undetectable levels (Dong et al, 1995).

Therefore, although the glucosidase inhibitors may have limited value as anti-viremics (in reducing viral DNA in the circulation), they have a novel, perhaps unique, contribution to make in reducing viral antigen levels in the circulation (antigen reduction). We suggest that there is compelling evidence in animals and people that therapeutic vaccination of HBV carriers with HBV glycoproteins can be enhanced when antigenemia is concomitantly reduced, and much of the stimulation of the cellular immune response was directed against the MHBs (preS2) antigen epitopes, we propose to exploit the antigen reduction properties of glucosidase inhibitors in partnership with a vaccine therapy. Although, in the case of HBV, glucosidase inhibition appears to impact MHBs (and possibly LHBs) secretion to a far greater extent than SHBs secretion, if there is an enhancement of a cellular immunological response to MHBs, it is expected that there will be cross benefit, since the same cells that express MHBs, express the other viral antigens. Thus, clearance or management of M producing cells will also result in clearance or management of HBV producing cells, in general.

E antigen negative patients. It is important to note that both interferon and lamivudine are indicated only for e Antigen positive HBV carriers. Indeed, a key milestone of clinical benefit and indication for discontinuation of therapy is “serological conversion” in which eAg becomes undetectable and anti-eAg antibodies appear. There is thus no therapy indicated for the eAg negative carrier, and most (if not all) antivirals in then pipeline likely to be approved for human use will also be intended for the eAg positive individual. This, despite the fact that most HBV carriers in the world are eAg negative. There is a growing understanding that eAg negative carriers are still at high risk for liver disease and as much as half of all hepatocellular carcinoma occurs in the eAg negative population (e.g. McMahon, et al, 2000).

Since glucosidase inhibitors target viral polypeptides and reduce MHBs antigenemia, they would be expected to be useful in an eAg negative, sAg positive population. In this sense, they are unusual amongst the antivirals in development.

The present invention is based upon the theory that glucosidase inhibitors are ideal complements to vaccine therapy, since vaccine therapy elicits cellular and humoral responses to preS as well as S domains and glucosidase inhibitors such as nonyl DNJ and NBDNJ: (1) selectively reduce the amount of preS domains that appear in the serum of chronically infected animals and medium of infected tissue cultures; (2) are orally available; (3) have been shown to be well tolerated in animals (rats, woodchucks, dogs) at the doses needed to achieve concentrations that reduce the amount of MHBs in the serum (e.g. Fischl, et al, 1994, Cox et al, 2001, and Block et al, 1998); (4) would be used for a limited period of time (months, rather than years); (5) can be used in clinical settings in which end points that can be quickly assessed will be used (reduction of M antigenenia, acquisition of humoral and cellular responses to glycoprotein epitopes); (6) are expected to complement existing therapies; and (7) would be of therapeutic benefit of to a currently underserved population—the e Antigen negative carrier.

The method according to the present invention therefore comprises administering to a subject a vaccine comprising a HBV and/or HCV virus antigen and administering to the subject a glucosidase inhibitor in an amount effective to inhibit the activity of a glucosidase enzyme associated with the endoplasmic reticulum of a cell in the subject.

In one aspect, the virus is selected from the group consisting of a hepadna virus, such as HBV, a flavivirus, a pestivirus, such as a Hepatitis C virus, a bovine viral diarrhea virus, a classical swine fever virus, a border disease virus, or a hog cholera virus. In another aspect, the membrane is selected from the group consisting of a membrane that surrounds the lumen of the endoplasmic reticulum and a membrane that surrounds a lumen of the Golgi apparatus.

In a preferred embodiment of the invention, the glucosidase inhibitor is 1,5-dideoxy-1,5-imino-D-glucitol or a derivative thereof selected from the group consisting of an N-alkyl, N-acyl, N-aroyl, N-aralkyl, and O-acyl derivatives.

The vaccines useful according to the present invention include, for example, vaccines produced against the HBV variant HBsAg protein or a fragment thereof (U.S. Pat. Nos. 5,639,637; 5,851,823; 5,989,865);

Also, HBV surfage antigens can be used as antigenic fragments in production of vaccines (see, e.g. U.S. Pat. No. 6,022,543). Shortly, the three known components of HBV particles differ in their relative amounts of the protein composition. The term “HBV” according to the present invention means any subtype of the virus, particularly adw, ayw, adr and ayr, described in the literature (P. Valenzuela, Nature Vol. 280, p. 815 (1979), Gerlich, EP-A-85 111 361, Neurath, EP-A-85 102 250). There are three monomers called the major protein with 226 amino acids, the middle protein with 281 amino acids, and the large protein with 389 or 400 amino acids, depending on the subtype ayw and adw, respectively. The large protein is encoded by the complete sequence of the pre-S1-, pre-S2- and S-regions, whereas the middle protein is derived from only the pre-S2- and S-regions, and finally the major protein from only the S-region (Tiollais et al., 1985; Nature, 317, 489; Dubois et al., 1980: PNAS, 77, 4549; McAlzer et al,, 1984: Nature, 307, 178).

The vaccines may include adjuvants, such as combination of immunostimulatory oligonucleotides having at least one unmethylated CpG dinucleotide (CpG ODN) and a non-nucleic acid adjuvant, such as alum or MPL. Examples of effective immunostimulatory oligonucleotides are described in, for example, U.S. Pat. No. 6,406,705.

Peptide antigens which are immunoreactive with sera from individuals infected with hepatitis C virus (HCV) include, but are not limited to peptides disclosed in U.S. Pat. No. 5,843,639.

Other examples of vaccines useful according to the present invention are described in Menne et al. (2002) Menne, s., J L Immunization with surface antigen vaccine alone and after treatment with LFMAU breaks humoral and cell mediated immune tolerance in chronic woodchuck hepatitis virus infection. J. Virol. 76: 5305-b 14.

Infections by viruses that require host cell glycosidase enzymes to synthesize and properly fold viral envelope glycoproteins can be treated by administering an inhibitor of those enzymes to the host cell. A target virus is any virus that acquires a component of its envelope in cooperation with internal cell membrane associated with the endoplasmic reticulum (ER). Preferred viruses are members of the flavivirus or pestivirus class.

By a “membrane associated with the ER” of a cell is meant a membrane which surrounds the lumen of the ER of the cell, a membrane which surrounds a lumen of the Golgi apparatus (GA), a membrane which surrounds the lumen of a vesicle passing from the ER to the GA, a membrane which surrounds the lumen of a vesicle passing from the GA to the ER, a membrane which surrounds the lumen of a vesicle passing from the GA or the ER to the plasma membrane of the cell, a membrane which surrounds the lumen of a vesicle passing from the GA or the ER to the nuclear membrane of the cell, or a membrane which surrounds the lumen of a vesicle passing from the GA or the ER to a mitochondrial membrane of the cell. It is contemplated that the methods of the invention are preferably applied to inhibiting the production of a virus that acquires any morphogenetic component by derivation from any of the internal membranes of the host cell.

By a “glucosidase enzyme associated with the ER” of a cell is meant a glucosidase enzyme which is embedded within, bound to the luminal side of, or contained within a membrane associated with, the ER of the cell. By way of example, mammalian .alpha.-glucosidase I and mammalian .alpha-glucosidase II are glucosidase enzymes associated with the ER of a mammalian cell.

A virus-infected animal cell which is treated according to the methods of the invention may be any cell that comprises a glucosidase enzyme associated with an internal membrane of the cell, preferably an enzyme associated with the endoplasmic reticulum (ER). Treatment of mammalian cells, including but not limited to human liver cells and bovine monocytes are particularly preferred.

Agents that exhibit an inhibitory effect on glucosidases are believed to do so because they are structural analogs of glucose. One of these agents is the imino sugar designated 1,5-dideoxy-1,5-imino-D-glucitol (alternately designated deoxynojirimycin), hereinafter “DNJ.” Numerous DNJ derivatives have been described. DNJ and its alkyl derivatives are potent inhibitors of the N-linked oligosaccharide processing enzymes, .alpha.-glucosidase I and .alpha.-glucosidase II (Saunier et al. (1982) J Biol Chem 257:14155-14161; Elbein (1987) Ann Rev Biochem 56:497-534). These glucosidases are associated with the endoplasmic reticulum of mammalian cells. The N-butyl and N-nonyl derivatives of DNJ may also inhibit glucosyltransferases associated with the Golgi.

Methods for treating a mammal infected with respiratory syncytial virus (RSV) using DNJ derivatives have been described (U.S. Pat. No. 5,622,972 issued to Bryant et al.). It is believed that DNJ exhibits its inhibitory effects on glucosidase because it is a glucose analog. However, Bryant discloses no mechanism by which DNJ derivatives exhibited the observed anti-RSV activity. RSV, a parnamyxovirus, acquires its envelope from the plasma membrane of an RSV-infected cell.

The use of DNJ and N-methyl-DNJ has also been disclosed to interrupt the replication of non-defective retroviruses such as human immunodeficiency virus (HIV), feline leukemia virus, equine infectious anemia virus, and lentiviruses of sheep and goats (U.S. Pat. Nos. 5,643,888 and 5,264,356; Acosta et al. (1994) Am J Hosp Pharm 51:2251-2267).

We have previously shown that human Hepatitis B virus (HBV) secretion from human hepatoblastoma cells in tissue culture is sensitive to inhibitors of the .alpha.-glucosidase activity in the endoplasmic reticulum (ER) under conditions that do not compromise host viability (Block et al. 1994). Hepatitis B virus (HBV) infected liver cells secrete infectious, nucleocapsid-containing virions as well as an excess of non-infectious “subviral” articles that do not contain DNA. All of these particles are believed to bud from an ER compartment or a post-ER compartment such as the intermediate compartment (Huovila et al. (1992) J Cell Biol 118:1305-1320; Patzer et al. (1986) J Virol 58:884-892). Inhibition of mature HBV egress is caused by inhibition of the activity of one or more of the glucosidase enzymes or glucosyltransferase enzymes normally associated with the endoplasmic reticulum (ER) of 2.15 cells, which are derived from HepG2 cells (Lu et al. (1995) Virology 213:660-665; Lu et al. (1997) Proc Natl Acad Sci USA 94:2380-2385).

Studies suggest that the anti-HIV properties of DNJ derivatives are the result of improper glycoprocessing of HIV envelope proteins, rather than direct inhibition of HIV budding from cells (Dedera et al. (1990) AIDS Res Hum Retr6vir 6:785-794; Fischer et al. (1995) J Virol 69:5791-5797; Taylor et al. (1994) Antimicrob Agents Chemother 38:1780-1787).

One derivative of DNJ, namely N-butyl-1,5-dideoxy-1,5-imino-D-glucitol (NBDNJ), prevents egress of the mature HBV virion from stable transfected HepG2 cells, but does not prevent egress of subviral particles (Block et al. (1994) Proc Natl Acad Sci USA 91:2235-2239). Thus, morphogenesis of HIV virions which are believed to bud through the plasma membrane, appears to be unaffected by the presence of NBDNJ. However, the infectivity of the virus particles released from HIV-infected cells exposed to NBDNJ is greatly reduced relative to HIV particles released from cells which were not exposed to NBDNJ (Dedera et al., supra Fisher et al., supra; Taylor et al., supra). These studies suggest that the anti-HIV properties of NBDNJ are the result of improper viral fusion of target cells, rather than direct irihibition of HIV budding from cells.

More recently we demonstrated the anti-viral effect of glucosidase inhibitors in a woodchuck animal model of HBV infection. In woodchucks chronically infected with woodchuck hepatitis virus (WHV), treatment with ER .alpha.-glucosidase inhibitors results in the disruption of the proper folding and transport of viral envelope glycoproteins and prevents the secretion of infectious enveloped virus (Block et al., 1998).

Most significantly and apparently different from the situation with HIV and RSV, inhibition of only modest amounts of glucosidase resulted in massive inhibition of HBV and BVDV secretion. This suggests that, unlike with HIV and RSV, etc., for viruses that bud from internal membranes, disruption of only a minority of envelope viral proteins is sufficient to inhibit secretion of the virus. This may be due to the fact that our evidence suggests that disrupted HBV and BVDV viral proteins act as “dominant negative” poisons of virus secretion and may themselves be considered the antiviral drug, as much as the drug itself.

ER alpha-glucosidases are responsible for the stepwise removal of terminal glucose residues from N-glycan chains attached to nascent glycoproteins. This enables the glycoproteins to interact with the ER chaperones calnexin and calreticulin, which bind exclusively to mono-glucosylated glyc proteins. Interaction with calnexin is crucial for the correct folding of some but not all glycoproteins, and inhibitors of the glucosidases can be used to specifically target proteins that depend on it. N-linked glycans play many roles in the fate and functions of glycoproteins. One function is to assist in the folding of proteins by mediating interactions of the lectin-like chaperone proteins calnexin and calreticulin with nascent glycoproteins. It is these interactions that can be prevented by inhibiting the activity of the alpha.-glucosidases with agents such as N-butyl-DNJ and N-nonyl-DNJ, causing some proteins to be misfolded and retained within the endoplasmic reticulum (ER). We have shown that the N-nonyl-DNJ-induced misfolding of one of the hepatitis B virus (HBV) envelope glycoproteins prevents the formation and secretion of virus in vitro and that this inhibitor alters glycosylation and reduces the viral levels in an animal model of chronic HBV infection.

The exquisite sensitivity of HBV to alterations in the envelope proteins induced by .alpha.-glucosidase inhibition and the fact that it is not necessary to inhibit the enzyme to any great extent in order to achieve the observed anti-viral effect, led us to speculate that the sensitivity of the virus may be due to the fact that it has to oligomerize and assemble the envelope in the ER where folding takes place. Unlike the situation with HIV and RSV, a few misfolded envelope proteins may be sufficient to disrupt the proper envelopment process and amplify the adverse effect the inhibitor has on virus assembly as compared to the effect it has on host cell proteins, which do not seem to be impaired at anti-viral inhibitor concentrations. Our mechanism studies led us to propose that other viruses which acquire their envelopes from intracellular membranes such as the ER would be equally sensitive to ER .alpha.-glucosidase inhibition, provided one or more of their glycoproteins depended on calnexin-mediated folding.

Although HBV and HCV have completely different life cycles, they have three things in common: They target the liver, they bud from the ER and other internal membranes and their envelope, glycoprotein(s) fold via a calnexin-dependent pathway. This prompted us to investigate whether the same inhibitors shown to have an anti-viral effect on HBV could inhibit HCV by the same proposed mechanism.

The two HCV envelope glycoproteins E1 and E2, which contain five or six and eleven N-linked glycosylation sites, respectively, both interact with calnexin during productive folding (Choukhi et al., 1998). Due to the lack of an efficient cell culture replication system the understanding of HCV particle assembly is very, limited. However, the absence of complex glycans, the localization of expressed HCV glycoproteins in the ER, and the absence of these proteins on the cell surface suggest that initial virion morphogenesis occurs by budding into intracellular vesicles from the ER. Additionally, mature E1-E2 heterodimers do not leave the ER, and ER retention signals have been identified in the C-terminal regions of both E1 and E2.

This led us to investigate the effect of glucosidase inhibitors on another ER-budding virus, bovine viral diarrhea virus (BVDV), the tissue culture surrogate of human hepatitis C virus (HCV). In the absence of a suitable cell culture system able to support replication of human HCV, bovine viral diarrhea virus (BVDV) serves as the FDA approved model organism for HCV (FIG. 1), as both share a significant degree of local protein region homology (Miller et al., 1990), common replication strategies, and probably the same sub-cellular location for viral envelopment. Compounds found to have an antiviral effect against BVDV are highly recommended as potential candidates for treatment of HCV.

BVDV, like HCV, is a small enveloped positive-stranded RNA virus and, like all viruses within the Flaviviridae, encodes all of its proteins in a single, long open reading frame (ORF), with the structural proteins in the N-terminal portion of the polyprotein and the non-structural or replicative proteins at the C-terminal end. The BVDV polyprotein has 6 potential N-glycosylation sites in the region encoding for the two heterodimer-forming envelope proteins gp25 (E1) and gp53 (E2), and 8 potential N-glycosylation sites in the region encoding for gp48 (EO), a hydrophilic secreted protein of unknown function. The structures of the oligosaccharides attached to any of these glycoproteins remain to be determined. BVDV proved to be even more sensitive to ER .alpha.-glucosidase inhibitors. This and the facts that the inhibitors used are preferentially taken up by liver-type cells in vitro and exhibit a prolonged retention in the liver in vivo give rise to the exciting possibility that glucosidase inhibitors could be used as broad based antiviral hepatitis agents.

Herein we describe the sensitivity of BVDV to glucosidase inhibition and discuss the possible reasons for ER-budding viruses being selectively dependent upon glycan processing. We have discovered that cytotoxicity resulting from exposure of mammalian cells in tissue culture to bovine viral diarrhea virus (BVDV) is prevented by addition of a glucosidase inhibitor to the tissue culture medium. The glucosidase inhibitors that were used in the examples below included a derivative of 1,5-dideoxy-1,5-imino-D-glucitol (DNJ), in particular, N-butyl-DNJ (NBDNJ). Moreover, inhibition of BVDV-induced cytotoxicity was achieved under conditions in which little, if any, toxicity toward host cells was observed to be mediated by NBDNJ. Because BVDV is an accepted tissue culture model of hepatitis C virus (HCV) (Henzler, H.-J. and K. Kaiser (1998) Nature Biotech 16:1077-1078), the compositions and methods described herein for inhibiting morphogenesis of BVDV are also useful for inhibiting morphogenesis of HCV.

Preferred are N-alkyl, N-acyl, N-aroyl, N-aralkyl, and O-acyl derivatives of DNJ. A derivative of DNJ, which is particularly preferred, is N-butyl-DNJ. Another preferred DNJ derivative is 1,5-dideoxy-1,5-nonylylimino-D-glucitol, which is herein designated N-nonyl-DNJ or NN-DNJ.

DNJ derivatives which have been described, for example in U.S. Pat. No. 5,622,972, include 1,5-dideoxy-1,5-butylimino-D-glucitol; 1,5-dideoxy-1,5-butylimino-4R,6-O-phenylmethylene-D-glucitol; 1,5-dideoxy-1,5-methylimino-D-glucitol; 1,5-dideoxy-1,5-hexylimino-D-glucitol; 1,5-dideoxy-1,5-nonylylimino-D-glucitol; 1,5-dideoxy-1,5-(2-ethylbutylimino)-D-glucitol; 1,5-dideoxy-1,5-benzyloxycarbonylimino-D-glucitol; 1,5-dideoxy-1,5-phenylacetylimino-D-glucitol; 1,5-dideoxy-1,5-benzoylimino-D-glucitol; 1,5-dideoxy-1,5-ethylmalonylimino-D-glucitol; 1,5-dideoxy-1,5-hydrocinnamoylimino-D-glucitol; 1,5-dideoxy-1,5-methylmalonylimino-D-glucitol; 1,5-dideoxy-1,5-butylimino-4R,6-O-phenylmethylene-D-glucitol; 1,5-dideoxy-1,5-phenoxymethyl)carbonylimino-D-glucitol; 1,5-dideoxy-1,5-ethylbutylimino-D-glucitol; 1,5-dideoxy-1,5-hexylimino-4R,6-O-phenylmethylene-D-glucitol; 1,5-dideoxy-1,5-(2-methylpentyl)imino-D-glucitol; 1,5-dideoxy-1,5-(3-nicotinoyl)imino-D-glucitol; 1,5-dideoxy-1,5-cinnamoylimino-D-glucitol; 1,5-dideoxy-1,5-(4-chlorophenyl)acetylimino-D-glucitol; and 1,5-dideoxy-1,5-(4-biphenyl)acetylimino-D-glucitol.

The compounds are used as the imino-protected species or the di- and tetra-acetates, propionates, butyrates, isobutyrates of the imino protected species.

Methods of synthesizing DNJ derivatives are known and are described, for example, in U.S. Pat. Nos. 5,622,972, 4,246,345, 4,266,025, 4,405,714, and 4,806,650, and U.S. patent application Ser. No. 07/851,818, filed Mar. 16, 1992.

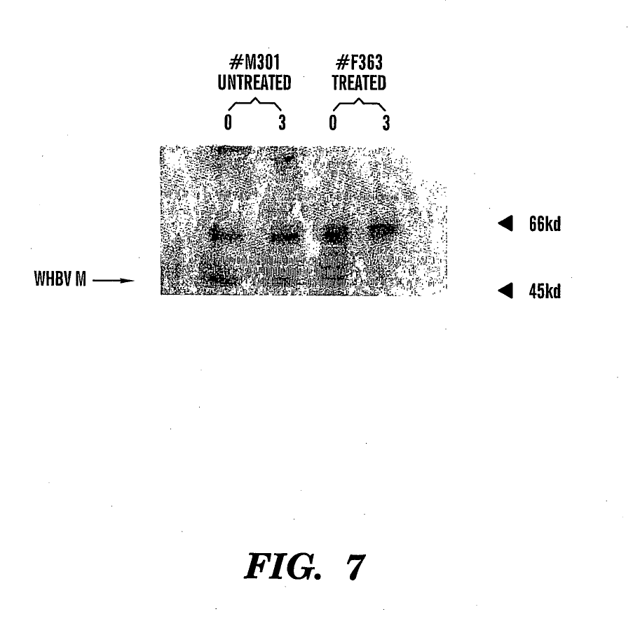

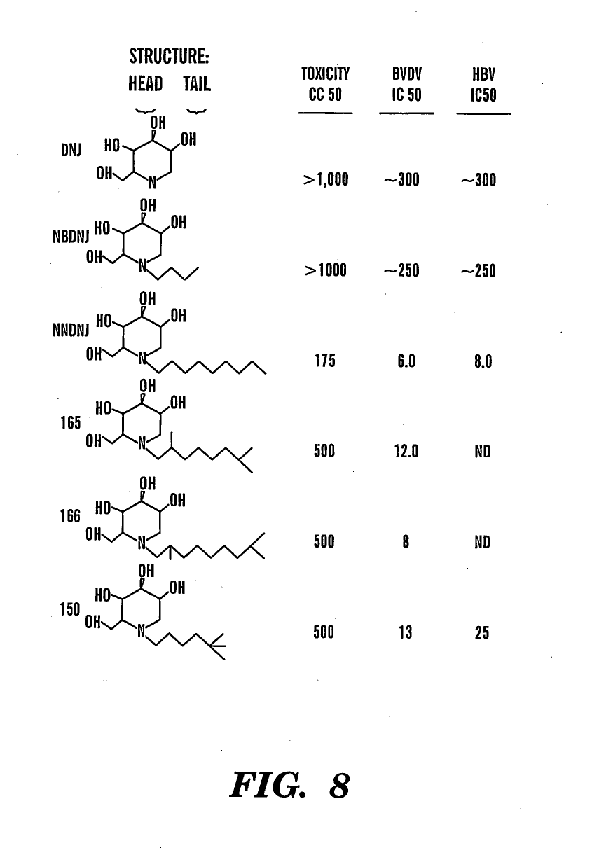

The substituents on the basic 1,5-dideoxy-1,5-imino-D-glucitol can influence the potency of the compound as an antiviral agent and additionally can preferentially target the molecule to one organ rather than another. For example, the N-butyl-substituted DNJ is less potent than the N-nonyl-subsituted-DNJ in inhibiting the intracellular production of BVDV virus (FIG. 1 and Example 2 in U.S. Pat. No. 6,465,487). Methods for comparing the potencies of various substituted compounds are provided in Example 1. The N-nonyl-substituted DNJ is preferentially taken up by liver cells (FIG. 7 and Example 7 in U.S. Pat. No. 6,465,487). Methods for determining preferential targeting properties of variously substituted DNJs is provided in Example 8 and FIG. 8 in U.S. Pat. No. 6,465,487.

The DNJ derivatives described herein may be used in the free amine form or in a pharmaceutically acceptable salt form. Pharmaceutical salts and methods for preparing salt forms are provided in Berge, S. et al. (1977) J Pharm Sci 6.6(1):1-18. A salt form is illustrated, for example, by the HCl salt of a DNJ derivative. DNJ derivatives may also be used in the form of prodrugs such as the 6-phosphorylated derivatives described in U.S. Pat. Nos. 5,043,273 and 5,103,008. Use of compositions which further comprise a pharmaceutically acceptable carrier and compositions which further comprise components useful for delivering the composition to an animal are explicitly contemplated. Numerous pharmaceutically acceptable carriers useful for delivering the compositions to a human and components useful for delivering the composition to other animals such as cattle are known in the art. Addition of such carriers and components to the composition of the invention is well within the level of ordinary skill in the art.

The methods of the invention may further comprise use of a DNJ derivative and a supplemental antiviral compound. The supplemental antiviral compound may be any antiviral agent, which is presently recognized, or any antiviral agent which becomes recognized. By way of example, the supplemental antiviral compound may be interferon-alpha, ribavirin, lamivudine, brefeldin A, monensin, Tuvirumab.™. (Protein Design Labs) Penciclovir.™. (SmithKline Beecham, Philadelphia, Pa.), Famciclovir.™. (SmithKline Beecham, Philadelphia, Pa.), Betaseron.™. (Chiron Corp.), Theradigm-HBV.™. (Cytel, La Jolla, Calif.), Adefovir Dipivoxil (GS 840, Gilead Sciences, Foster City, Calif.), Intron A.™. (Schering Plough), Roferon.™. (Roche Labs), beta interferon, BMS 200,475 (Bristol Myers Squibb), Lobucavir.™. (Bristol Myers Squibb), FTC (Triangle, Inc.), DAPD (Triangle, Inc.), thymosin alpha peptide, Glycovir (Block et al. (1994) Proc Natl Acad Sci 91:2235-2240), granulocyte macrophage colony stimulating factor (Martin et al. (1993) Hepatology 18:775-780), an “immune-cytokine” (Guidotti et al. (1994) J Virol 68:1265-1270), CDG (Fourel et al. (1994) J Virol 68:1059-1065), or the like.

The amount of antiviral agent administered to an animal or to an animal cell according to the methods of the invention is an amount effective to inhibit the activity of a glucosidase enzyme associated with the ER or other internal membranes in the cell. The amount of glucosyltransferase inhibitor administered to an animal or an animal cell according to the methods of the invention is an amount sufficient to inhibit the activity of a glucosylotransferase enzyme associated with the ER or other internal membranes in the cell. The term “inhibit” as used herein refers to the detectable reduction and/or elimination of a biological activity exhibited in the absence of a DNJ derivative compound according to the invention. The term “effective amount” refers to that amount of composition necessary to achieve the indicated effect. The term “treatment” as used herein refers to reducing or alleviating symptoms in a subject, preventing symptoms from worsening or progressing, inhibition or elimination of the causative agent, or prevention of the infection or disorder in a subject who is free therefrom.

Thus, for example, treatment of viral infection includes destruction of the infecting agent, inhibition of or interference with its growth or maturation, neutralization of its pathological effects, and the like. The amount of the composition which is administered to the cell or animal is preferably an amount that does not induce any toxic effects which outweigh the advantages which accompany its administration.

Actual dosage levels of active ingredients in the pharmaceutical compositions of this invention may be varied so as to administer an amount of the active compound(s) that is effective to achieve the desired therapeutic response for a particular patient.

The selected dose level will depend on the activity of the selected compound, the route of administration, the severity of the condition being treated, and the condition and prior medical history of the patient being treated. However, it is within the skill of the art to start doses of the compound(s) at levels lower than required to achieve the desired therapeutic effect and to gradually increase the dosage until the desired effect is achieved. If desired, the effective daily dose may be divided into multiple doses for purposes of administration, for example, two to four doses per day. It will be understood, however, that the specific dose level for any particular patient will depend on a variety of factors, including the body weight, general health, diet, time and route of administration and combination with other drugs and the severity of the disease being treated. It is expected that the adult human daily dosage will normally range from between about one microgram to about one gram, preferably from between about 10 mg and 100 mg, of the glucosidase inhibitor per kilogram body weight. Of course, the amount of the composition which should be administered to a cell or animal is dependent upon numerous factors well understood by one of skill in the art, such as the molecular weight of the glucosidase inhibitor, the route of administration, and the like.

Pharmaceutical compositions that are useful in the methods of the invention may be administered systemically in oral solid formulations, ophthalmic, suppository, aerosol, topical or other similar formulations. In addition to the glucosidase- or lucosyltransferase-inhibitor, such pharmaceutical compositions may contain pharmaceutically-acceptable carriers and other ingredients known to enhance and facilitate drug administration. Other possible formulations, such as nanoparticles, liposomes, resealed erythrocytes, and immunologically based systems may also be used to administer the glucosidase- or glucosyltransferase-inhibitor according to the method of the invention. Such pharmaceutical compositions may be administered by any known route. The term “parenteral” used herein includes subcutaneous, intravenous, intraarterial, intrathecal, and injection and infusion techniques, without limitation. By way of example, the pharmaceutical compositions may be administered orally, topically, parenterally, systemically, or by a pulmonary route.

These compositions may be administered according to the methods of the invention in a single dose or in multiple doses which are administered at different times. Because the inhibitory effect of the composition upon a virus endures longer than the inhibitory effect of the composition upon normal host cell protein glucosylation, the dosing regimen may be adjusted such that virus propagation is retarded while host cell protein glucosylation is minimally effected. By way of example, an animal may be administered a dose of the composition of the invention once per week, whereby virus propagation is retarded for the entire week, while host cell protein glucosylation is inhibited only for a short period once per week.

One advantage of administering these compositions is that they inhibit an enzyme of the host, rather than a viral function. It is well known that viruses are capable of mutating, whereby a viral function which is susceptible to inhibition by an antiviral agent mutates such that it becomes resistant to inhibition by the agent in progeny viruses. By way of example, the ability of the HIV virus to mutate such that it is rendered impervious to a particular anti-viral agent such as. AZT is well documented. The methods of the invention have the advantage that the composition used in the methods targets a host cell function employed by a virus as a part of its life cycle. This host function, namely glucosylation catalyzed by a host gluosidase associated with the host cell's ER or glucosyl transfer catalyzed by a host glucosyltransferase associated with the host cell's ER, is not subject to alteration brought about by a mutation in the genome of the virus. Thus, strains of the virus which are resistant to inhibition by the composition of the invention are unlikely to develop.

For therapeutic applications, the compounds may be suitably administered to a subject such as a mammal, particularly a human, alone or as part of a pharmaceutical composition, comprising the compounds together with one or more acceptable carriers thereof and optionally other therapeutic ingredients. The carrier(s) must be “acceptable” in the sense of being compatible with the other ingredients of the formulation and not deleterious to the recipient thereof.

The pharmaceutical compositions of the invention include those suitable for oral, rectal, nasal, topical (including buccal and sublingual), vaginal, parenteral (including subcutaneous, intramuscular, intravenous and intradermal), occular using eye drops, transpulmonary using aerosolubilized or nebulized drug administration. The formulations may conveniently be presented in unit dosage form, e.g., tablets and sustained release capsules, and in liposomes, and may be prepared by any methods well know in the art of pharmacy. (See, for example, Remington: The Science and Practice of Pharmacy by Alfonso R. Gennaro (Ed.) 20th edition, Dec. 15, 2000, Lippincott, Williams & Wilkins; ISBN: 0683306472.)

Such preparative methods include the step of bringing into association with the molecule to be administered ingredients such as the carrier which constitutes one or more accessory ingredients. In general, the compositions are prepared by uniformly and intimately bringing into association the active ingredients with liquid carriers, liposomes or finely divided solid carriers or both, and then if necessary shaping the product.

Compositions of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion, or packed in liposomes and as a bolus, etc.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface-active or dispersing agent. Molded tablets may be made by molding in a suitable machine a mixture of the powdered compound moistened with an inert liquid diluent. The tablets optionally may be coated or scored and may be formulated so as to provide slow or controlled release of the active ingredient therein.

Compositions suitable for topical administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets.

It will be appreciated that actual preferred amounts of a given compound used in a the therapy will vary according to the particular compound being utilized, the particular compositions formulated, the mode of application, the particular site of administration, the patient's weight, general health, sex, etc., the particular indication being treated, etc. and other such factors that are recognized by those skilled in the art including the attendant physician or veterinarian.

The vaccines useful according to the present invention include HBV vaccines that are well known to one skilled in the art of treating HBV infections. Infection with hepatitis B virus (HBV) is a serious, widespread problem but vaccines which can be used for mass immunisation are now available, for example the product ‘Engerix-B’ (SmithKline Beecham p.l.c.) which is obtained by genetic engineering techniques.

The cloning of genomes of Hepatitis B virions of different serotypes is well known in the art; see Miller et al., Hepatology, 9 (1989) page 322 and references therein. Dane particles which are hepatitis B virions and which are isolatable from infected patients have a diameter of about 42 nm. Each consists of an envelope comprising the hepatitis B surface antigen (HBsAg), a capsid (HBcAg), an endogenous polymerase and a DNA genome. A third polypeptide, ‘e’ antigen (HBeAg) is made by hepatitis B virus and found in solubilized form in serum.

Commercially available vaccines against HBV comprise Hepatitis B virus surface antigen (HBsAg) either in native or recombinant form. The authentic Hepatitis B virus surface antigen can be recovered from plasma of infected individuals as a particle of about 22 nm comprised of two proteins known as P24 and its glycosylated derivative GP28, both of which are encoded by the 226 amino acid coding sequence on the HBV genome known as the S protein coding sequence or HBV S-gene; see Tiollais et al, Nature, 317 (1985), page 489 and references therein. The complete amino acid sequence of, and nucleotide sequence encoding, HBsAg is given in Valenzuela et al, Nature, 280 (1979), page 815. The numbering system used by Tiollais et al. (loc cit.) to define nucleotide and amino acid positions is used herein.

Insertion of HBV S-gene coding sequences under the control of yeast promoters on expression vectors to enable expression of HBsAg in S. cerevisiae for vaccine production has been described by, for example, Harford et al in Develop. Biol. Standard. 54: page 125 (1983), Valenzuela et al., Nature 298, page 347 (1982) and Bitter et al., J. Med. Virol. 25, page 123 (1988). Expression in Pichia pastoris has also been described by Gregg et al, Biotechnoloay, 5 (1987), page 479 (see also European Patent Application Publication No. 0 226 846) as has expression in Hansenula polymorpha (see EP-A-0 299 108).

Vaccines may also be prepared from hybrid immunogenic particles comprising HBsAg protein as described in European Patent Application Publication No. 0 278 940.

Such particles can contain, for example, all or part or parts of the HBsAg precursor protein encoded by the coding sequence which immediately precedes the HBV-S gene on the HBV genome, referred to herein as the Pre S coding sequence. The Pre S coding sequence normally codes for 163 amino acids (in the case of the ay HBV sub type) and comprises a Pre S1 coding sequence and a Pre S2 coding sequence. The latter codes for 55 amino acids and immediately precedes the S protein coding sequence (see EP-A-0 278 940 for further details).

Antigenic subtypes of HBV are defined serologically and have been shown to be due to single base changes in the region of the genome encoding HBsAg (Okamoto et al., J. Virol., 1987, 74, 5463-5467). However, all known antigenic subtypes contain the ‘a’ determinant consisting of amino acids 124 to 147 of HBsAg. Antibody to the ‘a’ determinant confers protection against all subtypes. It has been shown by in vitro mutagenesis that the cysteine at position 147 and the proline at position 142 are important for the exhibition of full antigenicity of the ‘a’ determinant (Ashton et al, J. Med. Virol., 1989, 29, page 196).

Mc Mahon et al. have reported that substitution of arginine for glycine in the putative monoclonal antibody binding domain of HBsAg was found (as deduced by DNA sequence analysis) in a liver transplant patient treated with anti-HBsAg monoclonal antibody (Cold Spring Harbor Symposium on the Molecular Biology of Hepatitis B viruses, September, 1989). This result does not however provide any incentive to synthesise a variant HBsAg amino acid sequence or develop a vaccine composition based thereon.

In another report, children and adults were found with circulating hepatitis B surface antigen, indicating viral replication, despite the presence of specific antibody (anti-HBs) after immunisation with one of two licensed hepatitis B vaccines (Zanetti et al. Lancet, November 1988, page 1132). Analysis of the HBsAg with monoclonal antibodies revealed that the circulating antigen did not carry the ‘a’ determinant or that this determinant was masked. It was concluded that emergence of a variant of hepatitis B virus had been detected, possibly due to epidemiological pressure associated with immunisation in an endemic area of infection. The variant was, however, not characterised further.

From the work of Zanetti et al. it is clear that a potential disadvantage with presently available hepatitis B vaccines is that they may, at least in a host with a predisposing immunogenetic make-up, cause the appearance of an ‘escape mutant’, i.e. a replicating infectious virus that has mutated away from neutralising immunity. Such a variant virus clearly has the capacity to cause disease and may be assumed to be transmissible. The variant virus may therefore give rise to a serious immunisation problem since it is not effectively neutralised by antibodies produced by vaccines based on normal HBsAg.

A vaccine produced against an HBsAg protein or a fragment thereof displaying the antigenicity of HBV surface antigen, characterised in that the protein or fragment thereof comprises a modified ‘a’ determinant in which there is an amino acid other than glycine at position 145 of the HBsAg sequence (see, U.S. Pat. No. 5,639,637).

Other agents virus DNA replication and virion production, inhibiting the production of a hepatitis virus-induced antigen, inhibiting the production of a hepatitis virus-induced antigen: an alkyl lipid or alkyl lipid derivative (U.S. Pat. No. 5,770,584)

The antigen to which the cytotoxic T lymphocyte response is induced can be hepatitis B antigen HBc18-27 (U.S. Pat. No. 6,419,931)

### EXAMPLE

We have worked extensively on the biology, chemistry and mechanism of action of glucosidase inhibitors. The preliminary evidence provided here is intended to show (a) how glucosidase inhibitors selectively reduce HBV antigen levels secreted from infected cells, (b) demonstrate how the ability of these compounds to cause the accumulation of defective (possibly dominant negative) viral glycoproteins within the treated cell can be exploited for “low dose” therapeutic purposes, (c) demonstrate our advances in chemistry and (d) show how vaccination of woodchucks with WHV sub viral particles can elicit a beneficial immunological response.

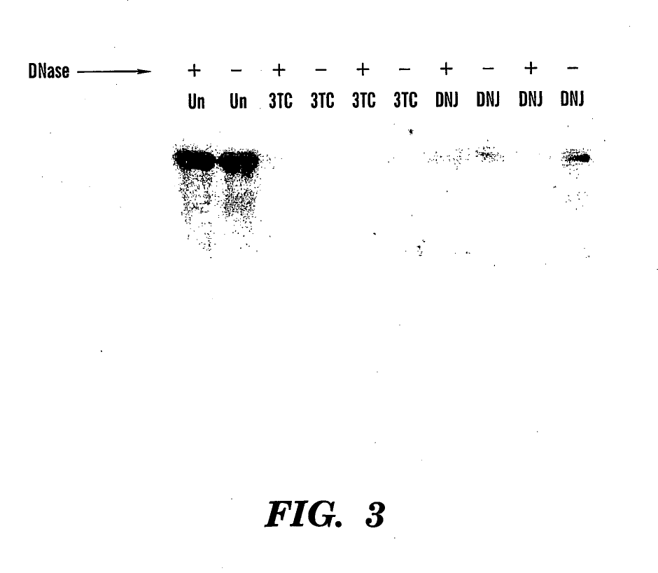

Inhibition of the ER a-glucosidases inhibits the secretion of enveloped HBV. The first glycan processing events are the removal of the terminal glucose residues in the ER by α-glucosidase I and II (FIG. 2). The α-glucosidase inhibitor N-butyldeoxynojirimycin (NB-DNJ) has been used to study the role of glucose processing in several proteins including human immunodeficiency virus (HIV-1) gp120 (Fisher et al., 1995) and the enzyme tyrosinase which is involved in melanin biosynthesis (Petrescu et al., 1996). Treatment of Hep G2.2.15 cells with NB-DNJ prevents the secretion of HBV viral particles (Block et al., 1994). An example of this is shown in FIG. 2. Hep G2 2.2.15 cells were either left untreated or treated with 3TC (3.5 μM) or the glucosidase inhibitor NB-DNJ (4.5 mM) and 7 days latter the amount of enveloped virus detected in the culture medium by a method that would differentiate between enveloped and un-enveloped virus (Wei et al., 1997). This method realizes upon the resistance of enveloped HBV to limited Proteinse K treatment (Wei et al., 1997). Briefly harvested viral partlces are collected and the total particle mix digested with Proteinase K. Enveloped particles are resistant and stay intact. Unenveloped particles are degraded and expose their nucleic acid. Addition of DNase results in the selective degradation of the en-enveloped HBV DNA. The remaining DNA is purfied by the addition of 1% SDS (final concentration) to remove the viral envelope, Proteinase K digestion and phenol cholorform extraction. This method was used as the glucosidase inhibitors only inhibit the secretion of enveloped viral DNA and not un-enveloped nucleocapid particles (Mehta et al., 2001). As FIG. 3 shows, treatment with DNJ inhibits the secretion of enveloped virus more effectively than un-enveloped virus. That is, although a substantial drop is seen with glucosidase inhibitor alone, a much greater drop is observed when one examines only the enveloped viral particle. Untreated cells secrete mainly enveloped particles and hence no change is observed via Proteinase K and Dnase treatment. These results indicate that glycan processing by glucosidase is required for the secretion of the HBV viral particle.

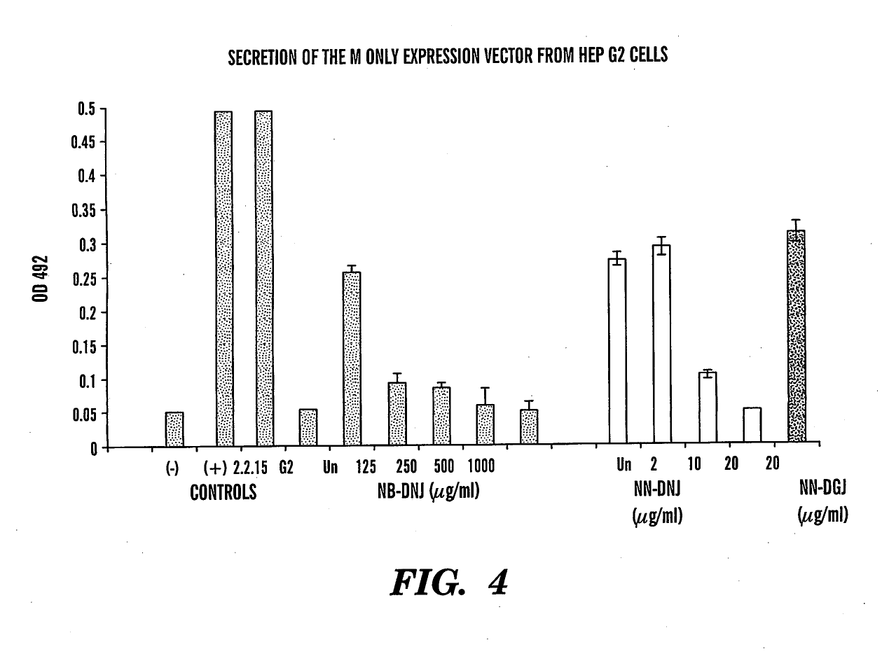

HBV M protein secretion is sensitive to glucosidase inhibition. Our previous work has determined that the secretion of HBV sub-viral particles containing the M protein was sensitive to glucosidase inhibition. (Mehta et al., 1997). That is, when Hep G2 2.215 cells were treated with the glucosidase inhibitor NB-DNJ, purified sub-viral particles appeared to lack the M protein. However, it was unclear if this was the result of the M protein's sensitivity to glucosidase inhibition or required some other viral factor. To test this hypothesis, an expression vector (pCMV-MS⁻X) directs the expression of HBV M, but no other viral protein, was created. This “M only” expression vector was then used to determine if the M protein itself, in the absence of other viral functions, was sensitive to glucosidase inhibition. In addition, an expression vector, unable to express “M”, but able to express “S” was made on the same plasmid backbone as the “M” only vector, as was used as a control (pCMV-M⁻SX). For these studies three imino sugars were used: N-butyl-DNJ, N-nonyl-DNJ and N-nonyl-DGJ. N-butyl-DNJ is our standard glucosidase inhibitor and has been used by several labs for over 10 years (Carlson et al.,1995, Block et al., 1994). N-nonyl-DNJ is an alkyl chain modification of N-butyl-DNJ and has been shown to only inhibit glucosidase 15-20% (Mehta et al., 2001; Durrantel, 2001). It is used here to demonstrate the extreme sensitivity of the HBV M protein to glucosidase inhibition. N-nonyl-DGJ is the galactose version of N-nonyl-DGJ and does not inhibit glucosidase and is used here as a negative control. Briefly, Hep G2 cells were transfected with the expression vectors pCMV HBV MS⁻X and pCMV HBV M⁻SX and subsequently seeded into fresh 6 well trays. The transfected cells were treated with the indicated concentrations of NB-DNJ, NN-DNJ or NN-DGJ (as a negative control) and three days latter the media analyzed for the presence of the HBV M or HBV S protein as described in Mehta et al, 2001. As FIG. 4 shows, cells that remained untreated secreted large amounts of M protein (untreated bar). In contrast, treatment with NB-DNJ or NN-DNJ prevented the secretion of the M protein. Even 10 μg/ml NN-DNJ, which, under these conditions, only inhibits glycan processing 15-20% (Jordan et al, 2002 and data not shown), has the ability to reduce the amount of M protein in the culture medium to almost undetectable levels. Lower concentrations of NB-DNJ, which have been shown to inhibit lower levels of glucosidase (Carlson et al., 1995), were also able to inhibit M protein secretion, and in other studies, it is clear that inhibition of glucosidase is necessary to commence M antigen secretion reductions. Fore example, concentrations of NN-DNJ which have no impact upon glycan inhibition, as determined by glycan analysis or α-1-acid-glycoprotein secretion, had no effect upon HBV M protein secretion (data not shown). In contrast to the effect of the glucosidase inhibitors on M protein secretion, the secretion of the HBV S protein was insensitive to either NB-DNJ or NN-DNJ providing further proof that the S protein is insensitive to glucosidase inhibition (data not shown; Block et al., 1994, Mehta et al., 1997, Werr & Prange, 1998). To determine if the secretion of the HBV M protein was prevented by another non-glucosidase mechanism, other compounds with the same alkyl side chain (tail) but altered imino sugar head groups, were synthesized. For example, NN-DGJ has the same chain length as NN-DNJ but is not a glucosidase inhibitor. Therefore, if NN-DNJ was inhibiting M protein secretion by some non-glucosidase mechanism, the other non-glucosidase inhibitor compounds should prevent the secretion of the M protein. However, as FIG. 4 shows, only glucosidase competitive inhibitors (NN and NB DNJs) but not NN-DGJ which does not inhibit glucosidase, reduces the amount of M detected in the medium.

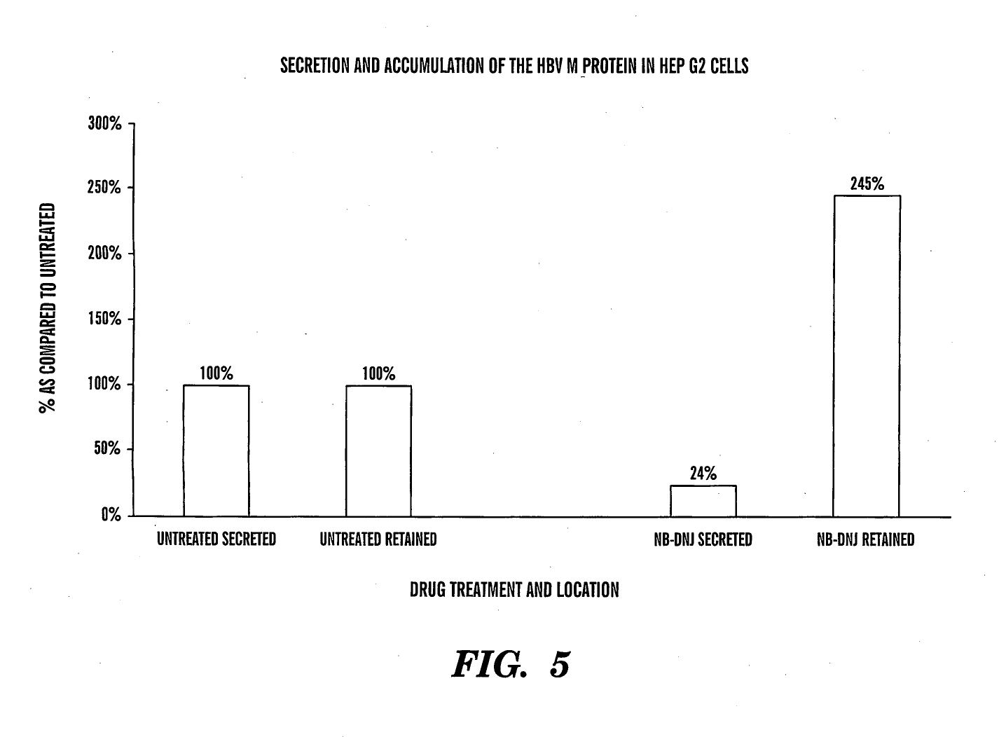

M protein accumulates in glucosidase inhibited cells. As stated, for glycoproteins, the processing of the initial oligosaccharide precursor from the Glc₃Man₉GlcNAc₂ glycoform to the Glc₁Man₉GlcNAc₂ glycoform in the ER can allow for interactions with chaperones such as calnexin and calreticulin (FIG. 2 & Ou et al., 1993). Calnexin, which binds only to glycoproteins containing Glc₁Man₉GlcNAc₂ structures, is thought to assist the folding of some, but not all glycoproteins (Helenius, 1995). The HBV M protein has been shown to interact with calnexin and more importantly, when this interaction is prevented, the M protein is not secreted. Thus, glucosidase inhibitors prevent the formation of the glycoform required for calnexin interaction and prevent the proper folding and secretion of the HBV M protein. Surprisingly, glucosidase inhibition causes the dramatic intracellular accumulation of intracellular M protein (Lu et al., 1997), rather then accelerating it's intracellular degradation, as might be expected for intracellular, detained, presumably unfolded proteins. An example of this intracellular accumulation is given in FIG. 5. Briefly, a T-75 flask of Hep G2 cells was transfected with the M only expression vector and the next day the cells divided equally into 6 well trays. One day later, the glucosidase inhibitor NB-DNJ (4.5 mM) was added. After 6 days of treatment (media changes occurred every two days) the cells and culture medium were removed and tested for the level of HBV M protein by ELISA as described earlier. Values were normalized to untreated controls. As this figure shows, treatment with the glucosidase inhibitor lead to a reduction in the amount of HBV M protein associated with the culture medium. In contrast, glucosidase inhibition leads to the retention of the HBV M protein. This accumulation peaks after 4 days of inhibition and remains constant thereafter. It is noted that our work has also shown similar results with enveloped HBV under these same conditions (Mehta et al, 1997).

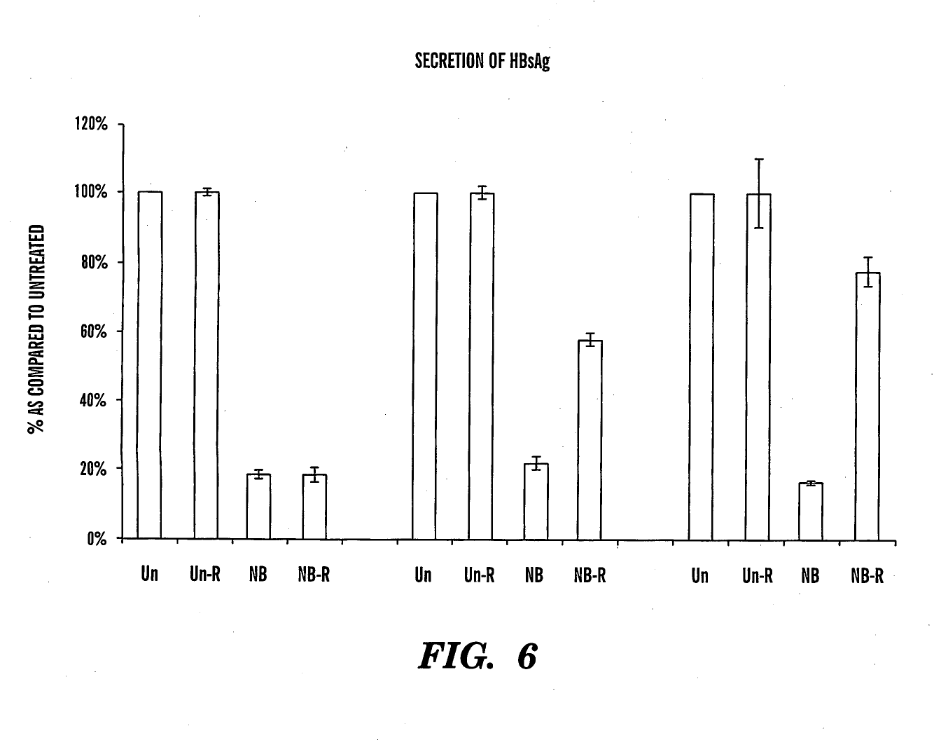

Glucosidase inhibitors have a prolonged effect on M protein secretion. As stated, glucosidase inhibitors such as NB-DNJ prevent the formation of Glc₁Man₉GlcNAc₂ glycoform that is required for the proper interaction with the ER chaperone calnexin (Ou et al., 1993; Werr & Prange, 1998). When the HBV M protein fails to interact with calnexin, evidence suggests that is misfolded and may act in a dominant negative manor (Lu at al., 1997; Mehta et al., 1997; Werr & Prange, 1998). As we have shown that the M protein accumulates in glucosidase-inhibited cells it was of interest to determine what effect this would have on further antigen secretion. Therefore a T-75 flask of Hep G2 cells was transfected with the M only expression vector and the next day the cells divided equally into 6 well trays. NB-DNJ was added at a dose that would repress M particle secretion. After four days (medium was changed two days after drug addition), the medium was removed and either fresh compound added or replaced with fresh media (labeled with -R). This is referred as the start of the rebound period. Subsequently medium was changed at one day later and than every 2 days for a total of 5 days after drug removal. The presence of sub-viral particles in the medium was detected using the Abbott Diagnostics Auszyme Monoclonal Diagnostic Kit as per manufacture's directions. As FIG. 6 shows, after 4 days of treatment, the glucosidase inhibitor leads to a substantial decrease in the amount of M protein in the culture medium. Surprisingly, when compound was removed, the level of M protein in the culture medium remained depressed until the 5^{th} day. This is true despite the fact that glucosidase enzyme function returns after less than 3 hours of removal of the competitive inhibitor, NBDNJ (not shown or see Tan et al, 1994). Work is in progress to determine why antigen secretion remains repressed long after glucosidase function has return. The sustained reduction is not due to an effect on cell number or viability (data not shown).

Glucosidase inhibition in vivo leads to the disappearance of the WHV M protein. Glucosidase inhibition in vitro leads to a decline in the amount of HBsAg secreted from HBV infected cells and a marked reduction of sub-viral particles containing the HBV M glycoprotein (Lu et al., 1997; Mehta et al., 1997). It was of interest to determine if the effect of glucosidase inhibition on M could occur in vivo. In the course of studying NNDNJ in woodchucks, we examined the WHV M profile in serum of treated woodchucks. There were very few samples available for study, but a first determination was encouraging. FIG. 7 shows an example, representative of all three animals studied, where woodchucks which have responded to drug treatment (demonstrated evidence of glycoprocessing inhibition) experience a disappearance of a polypeptide shown by N-terminal sequence analysis to be the WHV M protein. Briefly, serum from an untreated (M301) and N-nonyl-DNJ treated (F363) animal either pre-treatment or 3 weeks after treatment were partially purified as described in materials and methods and the M protein detected using an antibody directed against the pre-S2 domain of WHV M (a gift from William Mason). As FIG. 5 shows, a band of 46 kd, which is present in the untreated animal at 0 and 3 weeks and the treated animal at week 0, disappears at 3 weeks of N-nonyl-DNJ treatment. N-terminal sequence analysis of the 46 kd band (performed by Anthony Willis, MRC Unit, University of Oxford) missing in animal F363 at 3 weeks, has identified it as the WHV M protein. The size discrepancy between the WHV M protein and the HBV M protein is the result of extensive O-linked glycosylation in the WHV M protein that is not seen in the human version (Toll et al., 1998). Although the number of animals studied is small, the results are consistent with our prediction that glucosidase inhibition causes a reduction in M antigenemia. Clearly, more work needs to be done to confirm these data, and this is part of the proposal.

A large portfolio of glucosidase inhibitors are available in house. In our examination of glucosidase function in the morphogenesis of hepatitis B virus and hepatitis C virus, we have developed a portfolio of imino sugars based upon the DNJ head group. Deoxynorjirimycin (DNJ) is the prototype imino sugar with competitive inhibitory activity against the ER glycoprocessing enzymes glucosidases I and II (Tan et al., 1991). An example of these compounds (we have made more than 40, total) is shown in FIG. 8. Modification of the alkyl tail attached to this sugar head group has been previously reported to enhance both ability to inhibit glycan processing and virus production, in cell based assays (Tan et al, 1994; Durantel et al., 2001). The role of the side chain in enhancing activity is not fully understood but may be related to increasing the cellular uptake of the molecule or even functions in addition to glucosidase inhibition. FIG. 8 shows a subset of imino sugars that we have made and tested for their ability to inhibit either bovine viral diarrhea virus (BVDV; Ziztmann et al., 1999) or HBV. We have tested many of these compounds for their anti-BVDV activity and will screen these also for their ability to inhibit glucosidase as well as the secretion of the HBV M protein.

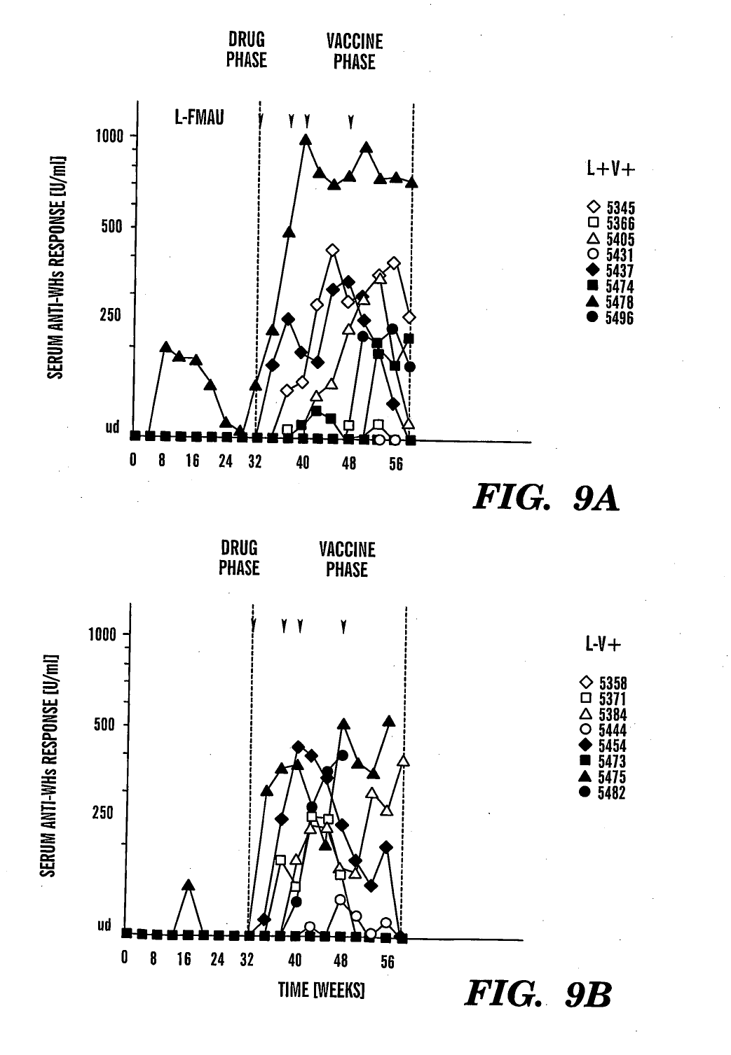

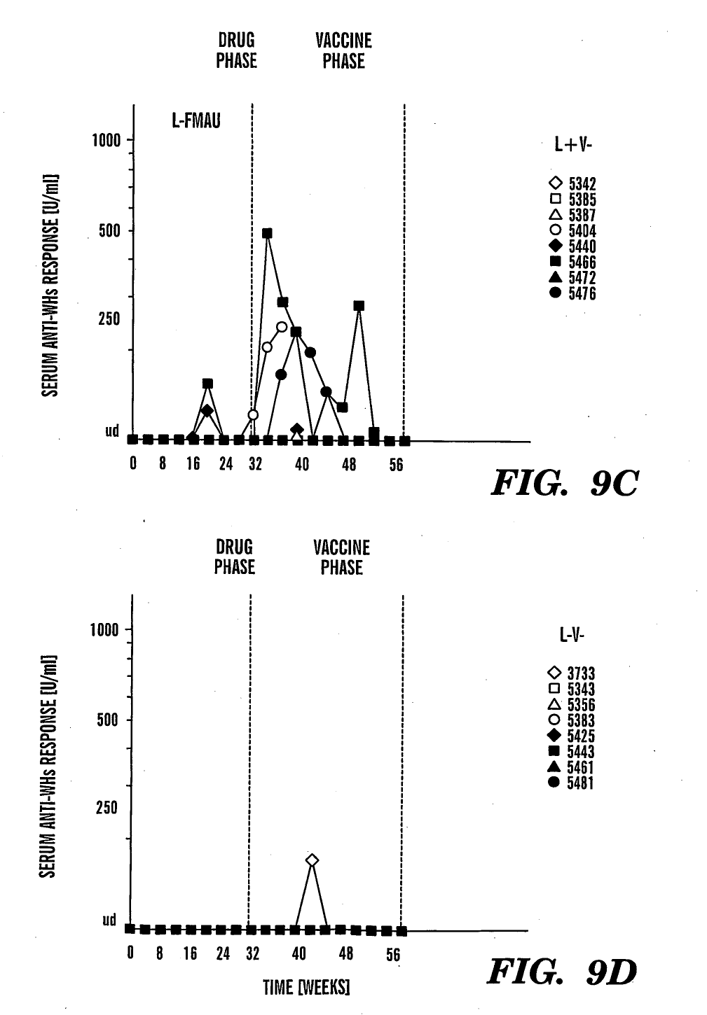

Therapeutic vaccination in combination with anti-viral treatment breaks humoral and cellular immune tolerance in chronic woodchuck hepatitis infection. Chronic HBV infection is characterized by defects in the immune response (Chisari, 2000; Menne and Tennant, 1999). In contrast, acute, self limiting HBV infection is characterized by a strong humoral and cellular immune response that both inhibits HBV replication and clears infected hepatocytes (Guidotti et al; 1999). Thus the development of a strong immune response to HBV may be the key to clearing the infection. Indeed, patients that respond favorably to interferon develop strong, broad, cellular immune responses to HBV. In addition, recent work has indicated that even treatment with anti-viral compounds that limit virus replication (3TC-lamivudine) can allow for the re-emergence of the host immune response (Boni et al., 1998, Boni et al., 2001). This is also seen in the woodchuck model of chronic HBV infection. The woodchuck model is a valuable model to study the basic pathogenesis of chronic HBV infection and in the development of anti-viral for the treatment of HBV. Woodchucks that have been treated with the anti-viral compound L—FMAU (1-(2-Fluoro-5-Methyl-β-L-Arabinofuranosyl)-Uracil) developed limited cellular and humoral immune responses as a result of decrease in both serum virus levels and in WHsAg. An example of this is shown in FIG. 9 and is described in greater detail in Menne et al., 2002. FIG. 9 shows the serum antibody response against WHsAg after treatment with L-FMAU and vaccination (L+,V+), L-FMAU alone (L+,V−), Vaccination alone (L−,V−) or untreated animals. As this figure shows, although vaccination alone stimulates the development of an anti-WHsAg response, the response is much greater in animals that received both anti-viral compound and vaccination. This is also true of the cellular response which developed against env, pol and core derived epitopes after anti-viral an vaccine treatment (data not shows, Menne et al., 2002). This response is thought to be the result of decreases in serum WHsAg levels and indicates that high viral antigen load may contribute to the immunogical tolerance that is seen in the chronic condition.

The strategy will be to first identify and validate the most appropriate glucosidase inhibitor to be used in the studies. This will be followed by the execution phase.

Determination of the best glucosidase inhibitor and analysis of glucosidase inhibition. Although our work, confirmed by others, has shown that efficient secretion of the HBV glycoprotein MHBs and LHBs requires the function of the ER glucosidase, the specific glucosidase inhibitor that would be most sensible to use in vivo studies is not entirely obvious. For example, micro molar concentrations of the nonyl derivative of DNJ (NNDNJ) has been shown in woodchuck experiments to reduce the amount of MHBs in the serum of chronically infected woodchucks to undetectable levels. However, NNDNJ has a dose limiting toxicity in dogs. Although this occurred at very high doses (more than 250 mg/kg), and we believe the toxicity was unrelated to the glucosidase inhibition mechanism), it seems wise to explore the possibility of alternative glucosidase inhibitors for the proposed study. Thus, Aim 1 will be to explore the possibility that other compounds, with less toxicity can be found. We would like to emphasize that NNDNJ has been shown to reduce MHBs antigenemia both in vitro and in vivo, at concentrations well below that expected to elicit the toxicity observed in dogs. It is therefore an attractive back up compound. Thus, although NNDNJ could be used in the proposed studies as a “stalking horse” and for a proof of concept, it will be “parked” (held in reserve) as we test other glucosidase inhibitors in our portfolio. Identification of the most appropriate glucosidase inhibitor

The criteria for nominating a glucosidase for further studies will be based upon biological and chemical standard as outlined below:

Biological Standard:

Compounds, from our portfolio of glucosidase inhibitors (see FIG. 8 for an example), will be tested for the ability, in tissue culture; to reduce the amount of MHBs secreted using two cell lines. Several compounds that are already in our portfolio and not shown in FIG. 8 that will be made and tested for their ability to inhibit M protein secretion (N-9-oxadecyl-DNJ, N-octyl-DNJ, and N-septyl-DNJ). The first cell line is a Huh7 derived line, Huh7-M, that constitutively expresses the MHBs “M” (and not other viral gene products) under the control of the CMV promoter. The second is the Hep G2 2.2.15 line. We feel it is important to use these two lines for the following reasons. Firstly, it is important to test the performance of any given compound on more than one cell line, to insure that efficacy and toxicity determinations are not cell line specific. The Huh7-M system provides a second cell line and to reassure that we do not choose a HepG2 specific compound. Also, as the only HBV envelope polypeptide it expresses is MHBs (see prelim. evidence) it also offers a simplified system for our testing, since a simple HBV envelope antigen capture assay can be used to measure polypeptide secretion. On the other hand, the 2.2.15 cell system is necessary since it expresses MHBs in the context of the other viral gene products and would most closely approximate an infected cell, in vivo. The assay for MHBs secreted from 2.2.15 cells, however, requires use of immunological reagents and methods that distinguish among the viral glycoproteins.

Compounds will first be screened for their ability to reduce HBV envelope secretion from Huh7-M cells. Promising leads will then be more rigorously tested in the 2.2.15 assay. Briefly, compounds or placebo (1 mg/ml dextrose), dissolved in aqueous solutions, will be incubated with the appropriate tissue culture, for 6 days, with changes of media (at days 2 and 4). Medium will be harvested on day 6 and the amount of MHBs as well as polypeptides representing the cell secreted products (alpha 1 antitypsin, as an example of a glycoportien and albumin, as an example of a non glycosylated) will be determined. Secretion of MHBs from Huh7-M will be determined by an ELISA, specific for HBV envelope proteins, using the Abbot Auzyme kit per manufacturers directions. The assay for MHBs from 2.2.15 cells will be by detection of the HBV specific polypeptides following sedimentation of culture medium by PEG precipitation and resolution through SDS-polyacrylamide gels and either silver staining or western blot analysis. The molecular weight of HBV specific polypeptides is very characteristic. In addition, a standard antigen capture assay for MHBs will also be performed using MHBs specific monoclonal antibodies (although this antibody is in short supply and hence will be used sparingly and only for confirmation).

The monoclonal antibody reagents for human antitrypsin and human albumin are commercially available, with a plentiful supply in hand, and distinguish between human and bovine sources. This is important since bovine serum will be used to culture cells. Therefore, the assay to detect albumin and antitrypsin, our controls for the impact of our compounds against cell secretion in general will be a straightforward western blot of polypeptides resolved in the culture medium.

Note that all monoclonal antibodies will be those that recognize their epitopes independent of glycosylation status of the protein.

Selectivity index and cytotoxicity assessment. The concentration of compound needed to reduce the amount of MHBs present in the culture medium (of MHBs producing cells) by 90%, relative to untreated or placebo treated controls will be considered to be the IC90 (inhibitory concentration, 90). The toxicity of the compounds to the tissue cultures will initially be determined the MTT assay, as described in (Lu et al., 1997). Briefly, the amount of MTT enzyme functional in the same sets of cells used for determination of the ability of a compound to reduce the secretion of MHBs (see above) will be measured. Thus, the impact of a compound upon cell viability will be assessed in the same cells in which MHBs reductions have been determined. The concentration of compound that results in a reduction of MTT activity (compared to untreated cultures) of 90% will be considered to be the CC90. The selectivity index will be expressed as the CC90 divided by the IC90. Using these criteria, as determined by assays on 2.2.15 cells under the conditions described in prelim. evidence, the selectivity index of NNDNJ is approximate 100. Thus, compounds will be considered attractive if they have a selectivity index of greater than 100.

Chemistry. For all the glucosidase inhibitors to be used in this proposal the starting material is DNJ. For the creation of alkyl tail compounds such as n-nonyl-DNJ, simple reductive alkylations with nonyl aldehydes reacted with DNJ will give rise to N-nonyl-DNJ. These reagents are commercially available. The synthesis of these compounds are familiar to our commercial collaborators, as described by Fleet et al (1988), van den Broek (1993) and Tan et al (1994). Ease of synthesis, and formulation and stability will also be considered in choosing the compounds to be used. That is, compounds that can be produced from commercially available stocks (such as DNJ imino sugar head groups, lactones, alkyl side chains) will be favored over those requiring multi step, laborious synthesis. Compounds that can be easily dissolved in aqueous solution, and remain stable at room temperature will be favored over those that are labile at room temperature and require organic solvents. A degree of hydrophobicity with the multi carbon side chain alkylated sugars is expected, however, although here are several formulation strategies that can reduce handling and use difficulty, such as preparation as the tartrate salt, as opposed to the free amine, etc. Some degree of discretion will be needed, here, since the value of a compound with outstanding efficacy and safety but poor formulation properties may prove to be better than compounds with excellent formulation but poor efficacy properties.

Detection of glucosidase inhibition. To confirm that the compounds being tested are glucosidase inhibitors, we will use an HPLC based assay to detect the changes in glycosylation that occur with glucosidase inhibition. This assay is extremely sensitive and can detect Pico-molar amount of glycan and is based upon the fact that inhibiting glucosidase function can prevent post-ER glycan processing and results in the accumulation and secretion of glycoproteins that contain tri-glucosylated glycan. The tri-glucosylated glycan (Glc₃Man₇GlcNAc₂) is an intermediate that accumulates as a result of glucosidase inhibition. Since cells with fully active glucosidases do not accumulate or secrete any detectable amount of tri-glucosylated glycan, the detection of tri-glucosylated glycan in the culture medium is used as a marker of glucosidase inhibition (Block et al., 1998; Mehta et al., 1998; Mehta et al., 2001). For these experiments, CHO cells will be used as they lack the Golgi-endomannosidase, a Golgi situated enzyme which can allow for glycan processing in the presence of glucosidase inhibitors. It should be noted that this enzyme only effect glycan processing in the post-ER complex and does not allow for intereactions with calnexin (Rabouille and Spiro, 1992). Briefly, CHO cells will be grown to confluency and subsequently treated with a concentration of compound that gives the greatest reduction of HBV M secretion. The removal and analysis of the glycan will be performed as before (Block et al., 1998; Mehta et al, 2001).

It is anticipated that there will be a correlation between those compounds that reduce M particle secretion and inhibition of glucosidase. The relative levels of glucosidase inhibition will be taken into consideration as well and be used to select compounds. That is, potent inhibitors of both glucosidase and M protein secretion will be given the highest priority for development.

Pharmaco-Kinetic and Toxicology in small rodents. Having identified the best glucosidase inhibitor for these studies and developed a compound serum detection assay, toxicology and pharmacokinetics analysis will be performed, first in rats and then in a 1 week study in woodchucks. These results will be used to guide us as to the appropriate dosing protocol to be used in the studies in Aims 3b and 4.

Woodchuck studies are expensive and resource demanding. Prior to performing in depth, multi-month experiments in woodchucks it is therefore necessary to get a sense of the toxicity of candidate compounds in small rodents to reduce the possibility of in vivo, dose limiting toxicity that was not anticipated by the in vitro toxicity studies. Once a range of compound concentrations is found that is well tolerated in the small rodents, short term pharmacokinetic analysis will be performed in woodchucks.

A standard 14-day repeated toxicity/dose range-finding study in rats will be performed on the candidate glucosidase inhibitor to be used in the woodchuck study. The study is designed to determine the dose range and maximum tolerated dose. This will be needed to set doses for the WHV/woodchuck efficacy animal study (in Aims 3 & 4).

Pharmacokinetics Analysis in rats. Groups of 9 female Sprague-Dawley rats will be given a single dose of the individual alkovir candidates by gavage, respectively. Clinical observations will be recorded at several intervals after dosing. Blood samples for pharmacokinetics will be collected predose, and at 5, 15, and 30 minutes post-dose, and at 1, 2, 4, 8, and 24 hours post-dose. Clinical observations will be recorded at several intervals after dosing. Blood samples for pharmacokinetics will be collected predose, and at 5, 15, and 30 minutes post-dose, and at 1, 2, 4, 8, and 24 hours post-dose. The samples will be analyzed in the HLPC assay (see above).

Rat Pharmacokinetic (PK) and toxicity study.. Sprague Dawley rats will be used. This study is based upon FDA guidelines for Pre-clinical Toxicity Testing of Investigational Drugs for Human Use (1968) as well as generally accepted guidelines for the testing of pharmaceutical compounds. Animals will be randomly assigned into one of the five groups. Animals will be individually caged and allowed to have free access to food and water. Dosing of animals will be twice daily by gavage.

For toxicity studies, each experiment will include: 1 vehicle control group, 1 control group of the α-glucosidase inhibitor N-nonyl-DNJ at 250 mg/kg bid, and 3 concentrations of the glucosidase inhibitor compound. These concentrations are likely to be (based upon our past experience with these compounds): 25 mg/kg bid, 250 mg/kg bid and 500 mg/kg bid), but may be less, depending upon in vitro efficacy and toxicity results. Animals will be randomly assigned into one of the five groups. Animals will be individually caged and allowed to have free access to food and water. Dosing of animals will be twice daily by gavage. Observation of changes in body weight and food consumption will be measured daily during the duration of the study. All surviving animals will be euthanized at the end of the 14 days and gross necropsy performed and organ weighted determined. All animals that die in the 14-day study will also be analyzed for gross necropsy and organ weights will be determined.

Animals will be subjected to daily “clinical” assessments noting general observable features such as feces output, struggling, salivation, skin/hair coloring, weight and, of course, morality. Animals will be sacrificed at the end of study and plasma levels and histopathological analysis will be performed by contract with a commercial pathology laboratory (such as White Eagle labs in Doylestown, Pa.—across the street from us—or The Hoyle Group in Frederick Md.). Gross (weight and morphology) and microscopic evaluation of organs (Kidney, liver, esophagus and epididymis will be performed, as is typical for imino sugar evaluation. These experiments will establish a safety profile of these compounds in small rodents.

Based upon the toxicology data obtained from a 14 day rat study on N-nonyl-DNJ, the toxicity with the alkovirs is predicated to be minimal. With N-nonyl-DNJ, toxicity primarily consisted of GI upset as a result of inhibiting the gut α-glucosidase enzymes involved in carbohydrate digestion. It should be noted that even though toxicity was observed, the maximum tolerated dose was determined to be 250 mg/kg bid. This was well above the amount required to achieve serum drug concentrations that were anti-viral. These data will be combined with those generated from oral dosing to obtain information on drug bioavailability, half life and dosing frequency.

The ability of the glucosidase inhibitor to reduce antigenemia in chronically infected woodchucks will be determined. Woodchucks chronically infected with hepadnavirus will either be fed, by oral gavage, placebo or glucosidase inhibitor. The amount of HBV glycoprotein in the serum, as a function of treatment, will be determined. Year 2-3.

Prior to performing a full vaccine—glucosidase inhibitor combination study in woodchucks, as outlined in Aim 4, a pharmacokinetic (PK) and then pilot “dose finding” study will be performed. The woodchuck PK study is important in determining the daily concentration/dose of glucosidase inhibitor that can be tolerated and necessary to achieve anticipated therapeutic levels. Although a general range of concentrations and dosing can be extrapolated from the small rodent experiments in Aim 1, the it will be wise to make a small investment of time and resources in a 1 week limited animal number woodchuck PK evaluation, since it is possible (given dietary and digestion differences) that the drug will behave differently in woodchucks.

Woodchucks. Experimental laboratory bred woodchucks, maintained in the College of Veterinary Medicine facilities of Cornell University will be used, under all appropriate Cornell University compliances. Chronic carriage of WHV results from the neonatal infection with WHV strain 7P1. Carriage is certified (confirmed) by serial sAg assays for envelope protein in the serum and by dot blot of serum for WHV specific DNA (Menne et al.,2002, Appendix).

Having determined a PK study in woodchucks and established a dose-serum concentration relationship, it will be important to determine the reasonable concentration of glucosidase inhibitor that reduces antigenemia will be conducted.

Woodchuck Pharmacokinetic Analysis. Three woodchucks will be used. Blood samples (approximately 1 mL) will be obtained from woodchucks while under anesthesia (ketamine/xylazine). The blood samples will be collected into heparinized tubes at approximately 0 (predose), 5, 15, 30 minutes, 1, 2, 4, 8, and 24 hours post-dose in the primary 1 week PK/tox study. These woodchucks will receive a single dose (i.e., half the total daily dose) on days of blood sampling for pharmacokinetics. The blood samples will be placed on wet ice immediately following collection. The samples will be centrifuged, and the plasma will be extracted, and plasma and packed red cells will be placed immediately in a −70° C. freezer. The frozen serum and cell samples will be packed in dry ice and sent via Federal Express overnight to TJU and our CRO.

Dose of glucosidase inhibitor that reduces M antigenemia pilot study. Twelve woodchucks, determined to be chronically carriers of woodchuck hepatitis virus (WHV strain 7P1) on the basis of sAg antigenemia and serum levels of WHV DNA (between 10× and 10× copies) will be used. SAg levels will be routinely determined by an antigen capture assay (Menne et al., 2002, Appendix) or, on occasion, western blot analysis of serum resolved through polyacrylamide gels, probing with rabbit antibody hyper immune for WHsAg or monoclonal antibody specific for MWHsAg, as in Block et al (1998) and Lu et al (2001), respectively. Examples of these assay are provided in the preliminary evidence. Animals will have been infected as neonates and under the care of our collaborator's laboratory (Dr. Bud Tennant) and maintained as in Menne et al., (2002) and generally as in Block et al (1998). 3 animals will be once or twice daily treated with placebo (dextrose) in fruit juice for the period of study. The other 9 animals will be placed into 3 dose groups. These animals will be given oral gavages of glucosidase inhibitor, dissolved in fruit juice, either once or twice a day for the study period. The doses to be used and frequency of administration will be determined by the compound half lives and toxicity information gleaned from the rat toxicity studies in aim 2. Although there is not an exact extrapolation that can be made from the rat data, our experience with imino sugar glucosidase inhibitors tested in rats and then woodchucks gives us confidence that an approximations can be made. It is likely that compounds will be used in the range of 3 to 24 mg/kg, aiming for single (or at most, twice) day dosing to achieve at least 1 micromolar serum levels.

WHV antigen levels. Although the level of circulating WHsAg is an important variable that can easily be determined by an ELISA, and will be determined in subsequent studies, for the experiments proposed here, it will first be necessary to determine the degree of reductions of LWHs and MWHs, as a function of glucosidase inhibitor. L and M can be distinguished by western blots and thus western blot assays as in Lu et al (2001). Briefly, 500 ul of woodchuck serum will be sedimented through 20% sucrose cushions, resolved in 12.5% SDS-PAGE gels and transferred to immobilon membranes (Millipore, Inc.) as in (Block et al., 1994). The membrane will be probed with mouse mAb specific to the WHV pre-S2 domain (a kind gift of William Mason, Fox Chase Cancer Center, Philadelphia, Pa., USA) followed by incubation with alkaline peroxidase conjugated rabbit anti-mouse serum (as in FIG. 6. Immunocomplexes were detected by Enhanced chemiluminescence (ECL; Amersham International, Buckinghamshire, UK) as per manufacturers instructions. Reductions in M (and possibly L) antigenemia will provide an independent assessment of the efficacy and activity of the glucosidase inhibitor. Reductions in antigenemia can be an independent measurement of benefit

Measurement of Serum WHV DNA levels. Briefly, serum will be taken in small aliquots and supplemented with 10 mM TRIS (pH 7.9), 10 mM EDTA (pH 8.0), and 10 mM MgCl₂. Proteinase K will be added to a final concentration of 750 μg/ml and the samples incubated for 1 hour at 37° c. After 1 hour, SQ1 Dnase (Promega, Madison, Wis.) will be added to each tube to a final concentration of 50 units/ml and incubated at 37° c. for 1 hour. After this incubation SDS will be added to a final concentration of 1% and more Proteinase K added to a final concentration of 500 μg/ml and the reaction allowed to proceed at 37° c. for 3-4 hours. DNA will be purified by phenol/chloroform extraction followed by isopropanol precipitation. DNA was separated by electrophoreses on a 1.0% agarose gel, transferred to a nylon membrane and probed with ³²p labeled HBV probes. Signals will than be detected via exposure to a phospho-image screen (Bio-Rad).

Compound concentration assay. Serum levels of glucosidase inhibitor will be determined by our HPLC assay.

Glycan processing inhibition. Glycan processing inhibition will be monitored by a quantitative HPLC assay that detects hyperglucosylated structures derived from N-linked glycans in the serum as in Block et al, 1998.. Glucosidase inhibitors prevent the processing of N-linked glycan in the endoplasmic reticulum, and the amount of unprocessed (hyper glucosylated) glycan in then serum has been used as a measurement or surrogate marker of glucosidase inhibition (Block et al., 1998). The degree to which glycan processing has been inhibited in treated animals will provide evidence that the glucosidase inhibitor used is having the intended effect upon its enzyme target and help validate predictions about the mechanism of action. It will also be important to correlate anti viral and immunological efficacy with glucosidase inhibitor concentration and degree of glycan processing inhibition.

Liver function tests. Animal viability and toxicity of the glucosidase inhibitor will be determined by the clinical observations (below) as well as serum analysis of a liver function test panel, as performed in the 1 week dose finding study.

Note that multiple serum dilutions will be used in each of the above assays to insure quantitative results applying the respective assays in their linear range of detection.

Clinical evaluation of woodchucks. Daily animal weight and other gross physical features (stool and urine output) will be recorded.

It is expected that a safe dose of glucosidase inhibitor that reduces the level of WHV M antigen will be found within the four weeks of study. The dose protocol that results in an at least 10 fold reduction in MWHsAg by week 4 will be considered as efficacious and sufficient for future studies, should the animals thrive (gain weight, remain physically similar to the untreated group). The western blot assay for MWHs will also detect LWHs, and it is possible that levels of the large antigen will decline in treated animals. Similarly, WHV DNA levels should also decline, relative to untreated and pretreatment controls, since monotherapy with glucosidase inhibitor should inhibit secretion of enveloped virus. However, the degree of reduction of WHV viremia, over this limited period of time, is not expected to greater than 10 fold. It will also be interesting to correlate serum glucosidase inhibitor concentrations with the degree of glycoprotein inhibition. We have found, in the past, that maximum levels of antiviral and anti-antigenemia reductions occur under conditions where less than 2% of all serum glycan is present in an unprocessed form (suggesting that modest glycan processing inhibition is sufficient to achieve maximum antiviral effects). Such results would be confirmatory and encouraging.

Determination if a glucosidase inhibitor can enhance the achievement of beneficial endpoints obtained with an HBV sAg containing vaccine.

Having selected an acceptable glucosidase inhibitor and validated and determined a safe and effective concentration that reduces HBV envelope antigenemia, woodchucks chronically infected with hepadnavirus will be either placebo treated or vaccinated with an sAg vaccine. Vaccinated animals will either be treated with the glucosidase inhibitor or left untreated. The levels of viremia, antigenemia, serological and lymphocyte profiles for reactivity against HBV specific epitopes will determined as a function of time and treatment.

It has been suggested that reductions of sAg in chronically infected woodchucks, enhances the efficacy of therapeutic vaccination. The value to enhancing therapeutic vaccination, of decreasing M and L levels, in the absence of significant reductions of s, is unknown. Since the source cells of S, M, and L (and all viral gene products, are the same hepatocytes (for the most part) it is possible that stimulation or enabling of a cellular response against any gene product, in principle, will have a beneficial effect upon.

This arm of the study will involve a total of 28 WHV chronically infected woodchucks split into 7 treatment groups as highlighted in FIG. 9. Animals will be randomly assigned to groups by WHV levels so that the average WHV level, determined 7 days prior to study start, is evenly distributed among all groups of animals. Animals with abnormally low WHV levels (<1×10⁸ genome equivalents/ml) will not be used in this study although such a population may ultimately be of interest, being more representative of an eAntigen negative group. The higher viremic entry critiera will be used, first, as our previous work had used animals that met this criteria (Block et al., 1998; Menne et al., 2002). Compound will be administered once or twice daily via oral administration, based upon results obtained in Aims 2 & 3. The dose volume will be 5 mL/kg in fruit juice. Vaccine will be the subunit preparation described in Menne et al., 2002. (Menne et al., 2002) and administered intra-muscularly. The day of dosing on the study will considered as Study Day 1. Study Day 1 dose levels will be calculated on a pretest body weight. Body weights will be taken weekly for dose administration. The length of time of glucosidase inhibition, prior to vaccination commencement will be determined by the experiments in Aim 2. It is expected that the length of time of glucosidase inhibition, prior to vaccination will be between 4-6 weeks (the time expected to be necessary to achieve glucosidase mediated antigen reduction). However, this will be empirically determined in Aim 2. Booster vaccination will be given at 4 week intervals after first vaccination.

TABLE 1
Experimental design with respect to the woodchucks and
their dosing_groups

| | | | | | Study |
|---|---|---|---|---|---|
| | | | | | Length |
| | | | | | (weeks), |
| | | | | Animals | plus 4 |
| Group | Drug | Drug | | per | week |
| Number¹ | Treatment² | Removed | Vaccine^{3,} | Group | follow-up |
| 1 | No Virus | N/A | NO | 2 | 30 |
| 2 | Virus | N/A | Yes | 4 | 30 |
| | infected | | | | |
| | But no drug | | | | |
| | treatment | | | | |
| 3 | Untreated | N/A | No | 4 | 30 |
| 4 | Untreated | N/A | Yes | 4 | 30 |
| 5 | Glucosidase | N/A | No | 4 | 30 |
| | Inhibitor | | | | |
| | (MTD) | | | | |
| 6 | Glucosidase | No | Yes | 4 | 30 |
| | Inhibitor | | | | |
| | (MTD) | | | | |
| 7 | Glucosidase | Yes | Yes | 4 | 30 |
| | Inhibitor | | | | |
| | (MTD) | | | | |
| ¹Virus and woodchucks: Experimental laboratory bred woodchucks, maintained in the College of Veterinary Medicine facilities of Cornell University will be used, under all appropriate Cornell University compliances. Chronic carriage | | | | | |
| # of WHV results from the neonatal infection with WHV strain 7P1. Carriage is certified (confirmed) by serial sAg assays for envelope protein in the serum and by dot blot of serum for WHV specific DNA. See Menne et al., 2002, Appendix). | | | | | |
| ²Compound: The glucosidase inhibitor will have been chosen from the work in Aims 1-3. It is expected to be active in the micromole range (in serum) and have a bioavailability and half life that will permit either once or twice daily, | | | | | |
| # oral gavage dosing. MTD is the maximum tolerated dose, to be determined. | | | | | |
| ³Vaccine: The WHsAg vaccine will be that described in Menne et al., 2002, Appendix. Briefly, this is a formalin inactivated subunit vaccine derived from rate zonal centrifugation and purification of 22 nM particles from serum of chronic | | | | | |
| # carrier woodchucks,. The subunits contain the epitopes of all three viral envelope proteins. | | | | | |
| ⁴The length of time of glucosidase inhibition, prior to vaccination commencement will be determined by the experiments in Aim 2. It is expected that the length of time of glucosidase inhibition, prior to vaccination will be between 4-6 | | | | | |
| # weeks (the time expected to be necessary to achieve glucosidase mediated antigen reduction) However, this will be empirically determined in Aim 2. Booster vaccination will be given at 2 week intervals after first vaccination. | | | | | |

Assays to be performed with justifications and frequency of assay. The over all goal of this project is to determine if glucosidase inhibitors can potentiate or enhance the efficacy observed with therapeutic vaccines, as reported by Menne et al (Menne et al., 2002) and shown in our preliminary evidence (provided by our collaborators). Therefore, the assays to be performed will be those used in the previous studies in which benefits from therapeutic vaccination of woodchucks were seen. In addition, assays will be performed that monitor the parameters (efficacy and biochemistry) of glucosidase inhibitors. All of the assays proposed are familiar to us and our collaborators, with reagents in house. Those conducting the assays will be blinded as to the treatment groups that they are examining.

TABLE 2
Approximate time table for the propose study.

| | | | | Weeks Of | | | | | | | | | | | | | Weeks Post | |
|---|---|---|---|---|---|---|---|---|---|---|---|---|---|---|---|---|---|---|
| | | | | Treatment | | | | | | | | | | | | | Treatment | |
| Event | Pre | 2 | 4 | 6 | 8 | 10 | 12 | 14 | 16 | 18 | 20 | 22 | 24 | 26 | 28 | 30 | 32 | 34 |
| Virus Load | X | X | X | X | | X | X | X | X | X | X | X | X | X | X | X | X | X |
| WHVsAg Analysis | X | X | X | X | | X | X | X | X | X | X | X | X | X | X | X | X | X |
| Hematology & | X | | | | | | | | | | X | | | | | | | |
| Chemistry¹ | | | | | | | | | | | | | | | | | | |
| Vaccine Treatment | | | | X | | X | | X | | X | | X | | X | | X | | |
| Anti-WHsAg response | X | | | | X | | X | | X | | X | | X | | X | | X | X |
| CTL Analysis | X | | | | X | | X | | X | | X | | X | | X | | X | X |
| Liver Biopsy | X | | | | | | | | | | | | | | | | | X |
| PK analysis | | X | | | | | | | | | | | | | | | | |

Note:
Exact time of vaccination will only be determined at the end of specific aim 2

Physical Examination, Virology and Biochemistry

Viability and animal health. Clinical observations will be performed and recorded once daily for morbidity and mortality. Further toxicology will be addressed via hematology, serum chemistry, and histology examination. A breakdown of the toxicity assays to be performed are given below in table 3. The possibility of untoward effects of the protocol must be determined. It is also essential to consider all biochemical and immunological endpoints in the context of general animal health to insure that decreases in viremia or antigenemia or other putative beneficial outcomes are not a secondary consequence protocol (compound) toxicity. Gross physical characteristics (weight, stool and urine out put and characterization, will be determined on a weekly basis as in Block et al, 1998. In addition, liver function tests (performed on samples collected monthly), hematology and chemistry (performed on pre, mid and end of treatment samples (as described in the table) and, for selected animals (at pre-dose, mid dose and end of treatment times), histology on wedge biopsy derived liver sections will also be performed for assessment of toxicity as well as efficacy (see Table 3).

Liver function tests:As in table 3, will be determined by the Cornell group in the weekly samples as a marker of liver viability.

Glycan processing inhibition. Glycan processing inhibition will be monitored by a quantitative HPLC assay that detects hyperglucosylated structures derived from N-linked glycans in the serum as in Block et al, 1998. Glucosidase inhibitors prevent the processing of N-linked glycan in the endoplasmic reticulum, and the amount of unprocessed (hyper glucosylated) glycan in then serum has been used as a measurement or surrogate marker of glucosidase inhibition ( Block et al., 1998). The degree to which glycan processing has been inhibited in treated animals will provide evidence that the glucosidase inhibitor used is having the intended effect upon its enzyme target and help validate predictions about the mechanism of action. It will also be important to correlate anti viral and immunological efficacy with glucosidase inhibitor concentration and degree of glycan processing inhibition.

WHV antigen levels. Serum sAg levels will be determined by two methods. The first, or primary assay, will be by an ELISA, as in Cote et al (1993), which can detect as little as 30 ng of antigen. However, the ELISA does not distinguish between S, M or L epitopes. It however, was the assay used by our collaborators in their previous studies and is simple and important to perform to insure overlapping end points between the study to be conducted and those in the past. S, L and M can be distinguished by western blots and thus western blot assays on woodchuck serum will be performed as in Aim 1. Reductions in M (and possibly L) antigenemia will provide an independent assessment of the efficacy and activity of the glucosidase inhibitor. Reductions in antigenemia be an independent measurement of benefit.

WHV virus levels in the serum. Performed as in Aim 2, except that dot blots (as in Menne et al, 2002) may be substituted for southern blot assays in the weekly experiments. Southern blot assays (described in Aim 2) will be performed on samples derived from every other week.

Intracellular WHV DNA levels. Wedge biopsies (limited times, see tables 2 and 3) will be performed by Dr. Tennant and colleagues and used for histology studies and intracellular WHV DNA examination. Briefly, 250 mg of solid tissue will be homogenized in 1 ml of homogenization buffer (100 mM NaCl, 1 mM EDTA, 50 mM Tris-base (pH 8.0), 0.5% NP40) using a dounce homogenizer (40-60 strokes). Samples will be clarified by centrifugation and the sample adjusted to 1% SDS and treated with 750 μg/ml of proteinase K for 4-6 hours at 37° C. DNA will be purified by phenol/chlorform extraction followed by isopropanol precipitation. DNA will be resolved through a 1.2% agarose gel and transferred to nylon membranes. Membranes will then be hybridized with a ³²P labeled probe containing the total WHV genome and developed by exposure to a phospho-image screen (Bio-Rad). Biopsy will be performed at three time points in the study, see Table III.

Evidence of humoral and cellular responsiveness. This was determined to be based upon the frequency of (percentage) of woodchucks in a given group that developed a “positive” response for any given time point. The frequency of positive samples was defined as the percentage of samples testing positive above the assay background during the interval of the study.

Humoral response. The presence of antibodies that recognize WHsAg will be determined by an ELISA (Cote et al, 1993) as used in Preliminary. Evidence. This assay is such that even WHs Abs complexed with antigen will be detected (Cote al, 1993). The presence of antibodies specific for either the L, M or S epitopes will be determined by a “semi” quantitative western blot in which the woodchuck serum to be tested is incubated with standard dilutions of WHV polypeptides that have been resolved through SDS PAGE. Standard western blot procedures are then followed using biotinylated protein G or, if necessary, second anti-woodchuck serum (Mehta and Block, unpublished).

Cellular Response. A woodchuck PBMC (peripiheral blood mononuclar cell) proliferation assay, developed by our collaborators, will be the basic screen used to detect evidence of cellular immunological recognition of antigens (Menne, 2002). It is similar to human cell PBMC assays (Ferrari et al., 1990). Briefly, woodchuck PBMCs are isolated from whole blood and stimulated in vitro as in (Cote, P. & J. Gerin, 1995,) Stimulated (dividing) cells are labeled with 2-3H) adenine and a stimulation index (SI) is determined by dividing the average sample cpm in the presence of stimulator by that in the absence. At least 5 replicates (and usually 7) are tested per sample. Assay to be performed at study beginning, at time of vaccination and every 2 weeks after the time of vaccination.

Stimulators of PMBC in the in vitro assay. Positive controls, such as conconavilin A, will be purchased commercially. 22 nM WHV sub viral particles (used in the preparation of vaccine) will be prepared from the serum of carrier woodchucks as in Menne et al. (2002, Appendix ) and incubated with PMBC cultures at approximately 1 ug/ml. Recombinant WHV peptides will also be produced by growth in E. coli and used at approximately 20 ug/ml. 1 ug/ml. recombinant core (intact and C-terminally truncated); eAg, “x”, are in house, with our collaborators. Synthetic WHV peptides representing cellular epitopes of core, preS1, S, preS2 and pol, as identified in Menne et al (2002) will be produced by commercial synthesis and used at between 1 and 20 ug/ml. The choice of “stimulators” to be tested is based upon the profile of cellular responsiveness seen by our collaborators in chronic carrier animals vaccinated with the WHV subunit vaccine, to be used here.

Examination of the possibility evidence that vaccine stimulated cellular responses to WHV will begin 8 weeks after vaccination, based upon our collaborators experience (see preliminary evidence ands Menne et al, appendix). Enhancement of this response mediated by glucosidase inhibitors will be most apparent at times before and immediately after 8 weeks of vaccination.

Reductions in antigenemia, particularly S antigenemia, and the appearance of s Ab are the best and most traditional serological endpoints of complete and protective and beneficial responsiveness. These endpoints have only been rarely been achieved by current approved and experimental therapies and it will be of interest to determine to what extent. If any, they achieved in this study.

However, the more conservative but still extremely important goal of the current study is to determine if animals receiving a combination of glucosidase inhibitor and vaccine more successfully achieve milestones than animals receiving either agent, alone. We are not looking for “synergy”, but rather “enhancement” or additively, much lower standards to meet. It will thus be necessary to have clear, unambiguous endpoints to allow comparisons from animal to animal and group to group.

Our bench mark of comparison will be animals treated with vaccine, alone (Group 2), vaccine plus glucosidase inhibitor (Group 6) and animals treated with placebo (group 3). In the study by Menne et al (Menne et al., 2002), and as alluded to in our preliminary evidence, in the absence of vaccine, there was no detectable anti-S WHs, and no detectable cellular response (stimulation above background in the PMBC assay) to WHV, “S”, preS or preS2 epitopes. On the other hand, of the vaccinated animals (a) 22% of the samples representing 6 different animals had greater than 100 Units of WHV antibody, although no animals receiving vaccine alone had detectable humoral activity against WHV at the end of study and (b) 43% of the samples had cellular responses in the PMBC assay to preS2 epitopes, with none sustained at the end of study. Therefore, we will be focused upon an increase in the titer of humoral response to S and M epitopes and an increase in the percentage of responsive animals. Sustained response, at the end of study, will be considered as evidence that successful potentiation has occurred. We do not expect animals on monotherapy with glucosidase inhibitor to develop significant humoral or cellular responses, but this remains to be seen, as well.

A statistical analytical approach will be taken in comparing immunological and/or virological responsiveness of one group relative to another or one group to itself, as a function of treatment. The approach will be that used by Menne et al, 2002 (see appendix). Briefly, standards of responsiveness for each test are set based upon background levels in the assay, and “stimulation indexes” (SIs) are assigned for a given sample. In the case of the cellular recognition of antigen, the frequency of “responsive” woodchucks is defined as the percentage of woodchucks in each group that demonstrated a positive response at one or more time points. These resulted in a value of a cumulative frequency value that could characterize the group and be used for statistical comparisons from group to group. The same logic will be used for other serological assays. As in Menne et al (2002), the Fisher's test for proportions will be used in group to group comparisons. One tailed test criteria will be used, significance parameters defined.

Taken together, the clearest evidence of enhanced responsiveness will be in the evaluation of cellular and humoral response, with decreased sAg and viremia, 4 weeks after the end of treatment and during the time in which drug has been withdrawn.

TABLE 3
Summary of Hematology and Clinical Chemistry
(performed on a monthly basis):

| Hematology | Clinical Chemistry | Histology |
|---|---|---|
| Total and relative | Total protein | Core Staining |
| differential leukocyte | | |
| counts | | |
| Erythrocyte counts | Albumin | WHsAg |
| | | Staining |
| Hemoglobin concentration | Globulin | HBV DNA |
| Hematocrit value | Albumin/Globulin ratio | Degree of |
| | | hepatitis |
| Mean corpuscular volume | Glucose | |
| Mean corpuscular | Cholesterol | |
| hemoglobin concentration | | |
| Platelet counts | Triglycerides | |
| | Total Bilirubin | |
| | Urea Nitrogen | |
| | Creatinine | |
| | Alanine Aminotransferase | |
| | (ALT) | |
| | Aspartate Aminotransferase | |
| | (AST) | |
| | Gamma-glutamyltransferase | |
| | (GGT) | |
| | SDH (Sorbitol | |
| | dehydrogenase) | |
| | Critical serum ion levels | |

### References

1. Allen, M. I., M. Deslauriers, C. W. Andrews, et al (1998) Hepatology. 27:1670-77.
2. Ausubel, F. et al (1987), Current Protocols in Molecular Biology, Ch. 4.10
3. Beasley, R. P. Cancer. 1988; 61: 1942-1956.
4. Block, T., Steiner, I., Spivak, J., McIntosh, M., Fraser, N. W. (1990). J. Virology. 64:3417-3426.
5. Block, T., Platt, F., Xuanyong, L., Gerlich, W., Foster, G., Blumberg, B., Dwek, R. Proc. Natl. Acad. Sci. (USA) 1994; 91: 2235-2239.
6. Block, T. M., Lu, X., Mehta, A., Blumberg, B., Tennant, B., Ebling, M., Korba, B., Lansky, D. M., Jacob, G. S., Dwek, R. A. Nature Medicine 1998; Vol 4, Num 5, 610-614.
7. Block, T. M. & R. Jordan (2002). Antiviral Chemistry and Chemotherapy. 12: 317-326.
8. Boni, C., A. Bertoletti, A. Penna, A. Cavalli, M. Pilli, S. Urbani, P. Scognamiglio, R. Boehme, R. Panebianco, F. Fiaccadori & C. Ferari (1998) J. Clin. Invest. 102: 968-975.
9. Boni C, Penna A, Ogg G S, Bertoletti A, Pilli M, Cavallo C, Cavalli A, Urbani S, Boehme R, Panebianco R, Fiaccadori F, Ferrari C. Hepatology. April 2001;33(4):963
10. Bruss, V., and Ganem, D. (1991) Proc. Natl. Acad. Sci. USA. 88: 1059-1063
11. Chisari, F. V. (2000) Am. J. Path. 156: 1118-1132.
12. Couillin, I., S. pol, M. Mancini, F. Driss, C. Brechot, P. Tiollais & M. L. Michel (1999) J. Infectious Dis. 180: 15-26.
13. Cox, T., Lachmann, R., Hollak, C., Aerts, J.,van Weely, S., Hrebicek, M., Platt, F. M., Butters, T. D., Dwek, R. A,. Moyses, C., Gow, I., Elstein, D., Zimran, (2000) The Lancet, 355, 1481-1485.
14. Cote, P. J., C. A. roneker, K. Cass, F. Schoedel, D. Peterson, B. C. tenant, F. de Noronha & J. L. Gerin (1993) Viral immunol. 6: 161-169.
15. Cote, P. & J. Gerin, (1995) Hepatology. 22: 687-699.
16. Doong S L, Tsai C H, Schinazi R F, Liotta D C, Cheng Y C, (1991) Proc Natl Acad Sci USA; 88(19): 8495-8499.
17. Durantel, D, Nichita, N. B, Carrouee-Durantel, S, Butters, T. D., Dwek, R. A, Zitzmann, N. (2001) J. Virol. 75: 8987-8998.
18. Ferrari C, Penna A, Bertoletti A, Valli A, Antoni A D, Giuberti T, Cavalli A, Petit M A. (1990) J Immunol. November 15;145(10):3442-9.
19. Fischl M A, Resnick L, Coombs R, Kremer A B, Pottage J C Jr, Fass R J, Fife K H, Powderly W G, Collier A C, Aspinall R L, Smith, S. L., Kowalski, K. G., Wallemark, C. B. J Acquir Immune Defic Syndr February 1994; 7(2): 139-47.
20. Gordien E, Rosmorduc O, Peltekian C, Garreau F, Brechot C, Kremsdorf D. Virol 2001; 75(6): 2684-91.
21. Guidotti, L. G., R. Rochford, J. Chung, M. Shapiro, R. Purcell & F. V. Chisari (1999) Science. 284: 825-829.
22. Guidotti,L. G., Morris,A., Mendez, H., Koch,R., Silverman,R. H., Williams, B. R. G., Chisari, F. V J. Virol., March 2002, p. 2617-2621 Vol. 76, No. 6
23. Grant, E. P. et al (1999) J. Exp. Med. 189: 195-205.
24. Hammond, C., I. Braakman, Helenius, A. Proc. Natl. Acad. Sci. USA 1994; 91: 913-917.
25. Hayashi, Y. & K. Koike (1989) J. Virol. 63: 2936-2940.
26. Heermann, K. H, and Gerlich, W. H. In: Mol. Biol of HBV, A Maclachlan, ed, CRC Press, Boca Raton Fla., USA 1992.
27. Hollinger, F. B. In Field's Virology, edited by B. N. Fields, D. M. Knipe et al., Raven Press, LTD. 1990: 2171-2236.
28. Hoofnagle, J. H., and Di Bisceglie, A. M. New Engl. Journ. Med. 1997;336: 347-356.
29. Hovanessian A, Rebouillat D, Mattei M G, Levy E R, Marie I, Monaco A P, Hovanessian A G., Genomics 1998 Sep. 15;52(3):267-77)
30. Jordan, R., Nikolaeva, O. V., Conyers, B., Wang, L., Mehta, A., Dwek, R. A., Block, T. (2002) (In Press).
31. Kakimi, K., Guidotti, L. G., Koezuka, Y., Chisari, F. V., (2000) J. Exp. Med. 921-930.
32. Kakimi, K., T. E. lane, F. V. Chisari & L. G. Guidotti, (2001) J. immunol. 167: 6701-5Kanazawa, T., S. nakamura, M. Momoi et al (2000) Inhibition of cytokinesis by a lipid metabolite, psychosine. J. Cell Biol. 15: 943-950.
33. Korba, A., B., Xie, H., Wright, K. N., Hombuckle, W. E., Gerin, J. L., Tennant, B. C., Hostetler, K. L., Hepatology 23: No.6, 958-963 (1996).
34. Liaw Y F. 2001. Antivir Chem Chemother;12:67-71
35. Lu, X., Mehta, A., Butters, T., Dwek, R. A., Block, T. M. Virology 1995; 213: 660-665.
36. Lu, X., Mehta, A., Dadmarz, M., Dwek, R. A., Blumberg, B. S., Block, T. M. Proc. Natl. Acad. Sci. (USA). 1997; 94: 2380-2385.
37. Lu, X., Lu, Y., Geschwindt, R., Dwek, R A & Block, T. M., (2001) DNA and Cell Biology, 20 (10) 647-656.
38. Lu, X. L. & Block, T. M., (2001) Enhancement of infection of HepG2 cells in culture, by pre-digestion of hepadnavims with V8 protease. Chapter in: Methods in Molecular Medicine. Hepatitis B protocols. J. Lau & R. Hammatake, editors, Humana Press, Totowa, N.J. (USA) (accepted)
39. Maschke, J., S. Menne, J. Jacob, E. Kreuzfelder, B. Tennant, M. Roggendorf, and H. Grosse-Wilde. 2001. Vet. Immunol. Immunopathol. 78:279-296.
40. Mason, William S., Cullen, J., Moraleda, G., Saputelli, J., Aldrich, C. E., Miller, D. S., Tennant, B. S., Frick, L., Averett, D., Condreay, L. D., Jilbert, A. R. (1998) Virology, 245:18-32
41. McMahon, B. J., L. Bulkow, A. Harspter, M. Snowball, A. Lannier, F. Sacco, E. Dunaway & J. Williams (2000) Hepatology. 32: 842-946.
42. Mehta, A., Conyers, B., Jordan, R., Butters, T., Dwek, R. A., Block. T. M. (2002) Submitted to Antimicrobial Agents and Chemotherapy.
43. Mehta A, Carrouee S, Conyers B, Jordan R, Butters T, Dwek R A, Block T M. 2001. Hepatology;33(6):1488-95
44. Mehta, A., Lu, X., Dwek, R. A., Blumberg, B., Block, T. M. Proc. Natl. Acad. Sci. (USA) 1997; 94, 1822-1827.
45. Menne, S., J. Maschke, T. K. Tolle, M. Lu, and M. Roggendorf. 1997. J. Virol. 71:65-74.
46. Menne, S., J. Maschke, R. Klaes, H. Grosse-Wilde, and M. Roggendorf. 1997. Cellular immune response of woodchucks to woodchuck hepatitis virus surface protein during acute WHV infection. pp. 453-457. In: Viral Hepatitis and Liver Diseases: Proceedings of the IX Triennial International Symposium on Hepatitis Viruses and Liver Disease. M. Rizzetto, R. Purcell, J. Gerin, and G. Verme (eds.), Edizoni Minerva Medica, Torino, Italy.
47. Menne, S., J. Maschke, T. K. Tolle, E. Kreuzfelder, H. Grosse-Wilde, and M. Roggendorf. 1997. Arch. Virol. 142:511-521.
48. Menne, S., J. Maschke, M. Lu, H. Grosse-Wilde, and M. Roggendorf. 1998. J. Virol. 72:6083-6091.
49. Menne, S. and B. C. Tennant. 1999. Nature Med. 10:1125-1126.
50. Menne, S., C. A. Roneker, J. L. Gerin, P. J. Cote, and B. C. Tennant (2002) J. Virol. 76:1769-1780.
51. Menne, S., C. A. Roneker, B. E. Korba, J. L. Gerin, B. C. Tennant, and P. J. Cote (2002) J. Virol. In press.
52. Moody, D. B., T. Ulrich, W. Muhlercker, D. Young, et al (2000) Nature. 404: 884-888.
53. Natori, T., M. Morita, K. Akimoto & Y. Koezuka, (1994) 50: 2771-2781
54. Nowak, M. A., Bonhoeffer, S., Hill, A. M., Boehme, R., Thomas, H. C., McDade, H. (1996) Proc. Natl. Acad. Sci. USA 93: 4398-4402
55. Ou, W. J., Cameron, P. H., Thomas, D. Y., Bergeron, J. M. Nature 1993; 364: 771-776.
56. Park, S. H. & A. Bendelac (2000) Nature. 406: 788-792.
57. Platt, F. M., Neises, G. R., Dwek, R. A., Butters, T. D. (1994) J. Biol. Chem. 269: 8362-8365
58. Platt, F. M & Butters, T. D. 2000. Biochemical Pharmacology, Vol. 56, pp 421-430.
59. Pollack, J. R., and Ganem, D., J. Virol., 1993; 67: 3254-3263.
60. Pollack, J. R., and Ganem, D., J. Virol., 1994; 68: 5579-5587.
61. Rehermann, R., D. Lau, J. H. Hoofnagle & F. V. Chisari (1996) J. Clin. Invest. 97: 1655-1665.
62. Robinson, W. S In Field's Virology, edited by B. N. Fields, D. M. Knipe et al., Raven Press, LTD., 1990: 2137-2169.
63. Rebouillat, D., and. Hovanessian, A. G (1999) J. Interferon and Cytokine Research (19) 295-308
64. Samuel, Charles E. (2001) Clinical Microbiology Reviews, Vol. 14, No. 4778-809
65. Schnena, M., D. Shalon, R. W. Davis & P. O. brown (270: 467470).
66. Schwartz, R H, 1992. Cell, 4, 1065-1079
67. Seifer M, Hamatake R K, Colonno R J, Standring D N. Antimicrob Agents Chemotherapy 1998; 42(12):3200-3208
68. Sells, M. A., Chen, M. L., Acs, G. (1987). Proc. Natl. Acad. Sci. USA. 84: 1005-1009.
69. Steel, L. ,F., T. Mattu, H. Heiberstriet, R. A. Dwek & T. M. Block (2001) Disease markers, in press
70. Shih, et al., (1989) Proc. Natl. Acad. Sci. USA, 86, 6223-6327
71. Su, Y., R. Lousch, N. Fraser, R. Chowhan & T. Block (1999) J. Virology (73:4171-4180)
72. Tan, A., L. van den Broek, J. Bolscher et al (1994) Glycobiol. 4: 141-149
73. Tan, A., van den Broek, L., van Boeckel S., Ploegh, H., Bolscher, J. J. Biol. Chem. 1991; 266: 14504-15410.
74. Tennant, B. C. & J. L. Gerin The woodchuck model of hepatitis B virus infection. In: The Liver: Biology and pathobiology. 3rd edition. I. M. Arias, N. Faustto, W. B. Jacoby, J. L. Boyer & D. A. Schacter & D. A. Shafritz, Eds. Raven press, Publisher (NY, N.Y.) pp. 1455-1466 (1994).
75. Terrault N, and Ma M. (2001) Hepatology; 33(6):1544-6.
76. Tipples, G. A., Ma, M., Fischer, K. P., Bain, V. G., Kneteman, N. M., and Tyrrell, D. L. J., Hepatology 24: 714-717 (1996).
77. Van der Broek, L. A., D. J. Vermaas, B. Heshkamp et al (1987) Recl. Trav. Chim. Pays-Bas. 112: 82-94.
78. Webster, G., A. Bertoletti (2001) Molecular Immunology. 38: 467473.
79. Wei, Y., Tavis, J. E., Ganem, D., J. Virol. 1997; 70: 6455-6458.
80. Werr, M., and Prange, R. (1998) J. Virol. 72: 778-782.
81. Wieland, S. F., L. Guidotti & F. V. Chisari (2000) J. Virol. 74: 4165-4173.
82. Wong, D. C., J. W. K. Shi, R. H. Purcell, J. L. Gerin & W. T. J. Clin. Microb. 15: 484-490 (1982).
83. Xu, Z., Bruss V., Yen T. S. J Virol. 1997 ;71(7):5487-94.
84. Zitzmann, N., Mehta, A. S., Carroueé, S., Butters, T. D., Platt, F. M., McCauley, J., Blumberg, B. S., Dwek, R. A., Block, T. M., Proc. Natl. Acad. Sci (USA), 1999; 96 (21): 11878-11882.

The references cited herein and throughout the specification are herein incorporated by reference in their entirety.

1. A method of treating a subject infected by a virus which acquires its envelope from a membrane-associated with the intracellular membrane comprising:
a) administering to the subject an antiviral vaccine comprising an antigenic fragment of the virus; and
b) administering to the subject an effective amount of an agent which inhibits morphogenesis of the virus and a pharmaceutically acceptable carrier.

2. The method according to claim 1, wherein the virus is characterized by acquiring its envelope from a membrane associated with the ER of a virus-infected cell.

3. The method according to claim 1, wherein the agent is a glucosidase inhibitor and the effective amount is an amount effective to inhibit the activity of a glucosidase.

4. The method according the claim 1, wherein the subject is human.

5. The method according to claim 1, wherein the virus is hepatitis B virus.

6. The method according to claim 1, wherein the virus is hepatitis C virus.

7. The method according to claim 5, wherein the antigenic fragment is HBV HBsAg protein or a fragment thereof.

8. The method according to claim 1, wherein the agent is selected from the group consisting of N-alkyl derivative of a 1,5-dideoxy-1,5imino-D-glucitol.

9. The method of claim 8, wherein the derivative is N-nonyl-1,5-dideoxy-1,5-imino-D-glucitol.
